(19) United States
(12) Patent Application Publication
(10) Pub. No.: US 2021/0121480 A1
(43) Pub. Date: Apr. 29, 2021

(54) STABLE PHARMACEUTICAL COMPOSITIONS CONTAINING ESTRADIOL AND PROGESTERONE FOR ORAL ADMINISTRATION

(71) Applicant: SLAYBACK PHARMA LLC, Princeton, NJ (US)

(72) Inventors: Vijayabhanu Prattipati, Hyderabad (IN); Venkateshwar Reddy Keesara, Hyderabad (IN); Naga Venkata Durga Prasad Ketha, Hyderabad (IN); Hanimi Reddy Bapatu, Hyderabad (IN)

(73) Assignee: SLAYBACK PHARMA LLC, Princeton, NJ (US)

(21) Appl. No.: 17/078,702

(22) Filed: Oct. 23, 2020

(30) Foreign Application Priority Data
Oct. 23, 2019 (IN) 201941043071

(51) Int. Cl. A61K 31/57; A61K 31/565; A61K 9/48

(52) U.S. Cl. CPC A61K 31/57; A61K 31/565; A61K 9/4858; A61K 9/4833; A61K 9/4825

(57) ABSTRACT

The present application relates to stable pharmaceutical compositions for oral administration comprising a progesterone alone, or optionally in combination with an estradiol. The pharmaceutical compositions may further include a solubilizing agent, a surfactant and optionally other pharmaceutical acceptable excipients. Preferably, the estradiol does not precipitate for at least 12 months, and the pharmaceutical compositions are stable for at least 12 months under normal storage conditions at room temperature. Also, when the pharmaceutical compositions are stored for 6 months at 40° C./75% relative humidity (RH), the level of Impurity-M in the composition is less than about 0.2% (w/w) as measured by HPLC. The pharmaceutical compositions may be used for the treatment of moderate to severe vasomotor symptoms due to menopause in post-menopausal women.

### CROSS-REFERENCE TO RELATED APPLICATIONS

This application claims foreign priority to Indian Application No. IN 2019/41043071, filed on Oct. 23, 2019, which is incorporated herein by reference in its entirety.

### FIELD OF THE INVENTION

The present application relates to stable pharmaceutical compositions comprising progesterone or a pharmaceutically acceptable salt thereof, optionally, in combination with estradiol or a pharmaceutically acceptable salt thereof, for oral administration, as well as to methods for preparing such pharmaceutical compositions.

In particular, certain aspects relates to stable oral compositions comprising estrogen and/or progesterone, wherein the compositions further comprise a solubilizing agent comprising a long-chain oil, and a surfactant, preferably wherein the solubilizing agent and the surfactant are present in the composition in a weight ratio of 50:50 to 99:1.

The pharmaceutical compositions may be used in methods for the treatment of moderate to severe vasomotor symptoms due to menopause, for preventing endometrial hyperplasia in non-hysterectomized postmenopausal women who are receiving conjugated estrogen, or for treating secondary amenorrhea in a human patient.

### BACKGROUND OF THE INVENTION

Hormone replacement therapy (HRT) is a medical treatment that involves the use of one or more of a group of medications designed to increase or supplement hormone levels in women who lack adequate hormone production. It can mitigate and prevent symptoms caused by diminished circulating estrogen and progesterone hormones.

HRT can mitigate and prevent symptoms caused by diminished circulating estrogen and progesterone hormones, regardless as to whether the subject is pre-menopausal, peri-menopausal, menopausal or post-menopausal. However, specific disease states can exist during each stage of menopausal progression.

Many postmenopausal women are treated with hormone therapy (HT) in an attempt to alleviate symptoms of menopause, which are primarily hot flashes, night sweats, and vaginal atrophy, and also to prevent osteoporosis. The postmenopausal period has been considered an endocrine-deficient state, and HT can help restore the premenopausal endocrine milieu. Estrogen is the principal hormone used to treat postmenopausal symptoms. A variety of estrogenic preparations are available, including the natural endogenous estrogen, 17β-estradiol. To prevent endometrial hyperplasia from the effects of exogenous estrogens in women with a uterus, a progestogen is used either continuously combined or sequentially with the estrogens. The progestogens available for therapeutic use are synthetic progestogens (progestins), and also natural progestogen (progesterone).

During the menopause transition, decreases in estrogen may cause women to experience vasomotor symptoms (VMS) and genitourinary symptoms of menopause. Vasomotor symptoms (VMS) have been shown to negatively affect quality of life, increase sleep disturbances, impair social relationships, interfere with mood, and decrease work productivity, and are especially bothersome when severe. For example, women with moderate to severe VMS have increased frequency of night time wakefulness and chronic insomnia. They are also more than three times more likely to report impaired quality of life than those with mild or moderate VMS. Women with moderate to severe VMS were 2.8 times more likely to have moderate to severe depressive symptoms than women with no or mild VMS.

Estrogen therapy has been successfully used for several decades for the management of these menopausal symptoms. However, the prolonged use of unopposed estrogens increases the risk of endometrial hyperplasia and endometrial cancer. In women with a uterus, the addition of a progestin to estrogen therapy has been shown to protect the uterus from estrogen-induced endometrial hyperplasia and endometrial cancer.

Estradiol is the predominant estrogen hormone produced by the human ovaries during the first half or follicular phase of the menstrual cycle. Estradiol is also the principal hormone which maintains bone density, reduces vasomotor symptoms, and maintains the normal structure of the female genital organs following menopause.

Progesterone is the principal hormone of the second half, or luteal phase of the menstrual cycle. Among other functions, progesterone acts as an estrogen antagonist. For example, it is well known that prolonged administration of estrogen can result in endometrial hypoplasia and even uterine cancer, whereas progesterone appears to block this unwanted effect of the endometrium.

It is desirable to use estradiol and progesterone to treat a variety of endocrine disorders, and also for use as a contraceptive. However, it is well known that neither of these compounds are suitable for oral administration due to the manner in which they are absorbed from the digestive system. In particular, these steroidal hormones are carried by the portal system to the liver, where they are rapidly metabolized into inactive metabolites. Consequently, effective oral administration has required excessively high dosage levels to compensate for the metabolic breakdown of these compounds.

While chemical modifications have resulted in enhanced oral activity of these steroidal hormones, they have also resulted in undesirable side effects. In particular, the synthetic progestins are well known for their side effects, which include masculinization and adverse effects on cholesterol levels, triglyceride levels, and high-density lipoprotein levels. In addition, synthetic progestins may also cause fluid retention and depression as significant side effects in contrast to natural progesterone.

Bio-identical hormones, which are identical in chemical structure to the hormones naturally produced by human bodies can be used and are often referred to as natural hormone replacement therapy, or NHRT. The term “bioidentical hormone” refers to exogenous hormone products that are chemically similar to endogenous hormones such as estradiol and progesterone. These natural or bio-identical hormones are formulated from various ingredients to match the chemical structure and effect of estradiol, estrone, or estriol (the 3 primary estrogens) as well as progesterone that occur naturally in the human body (endogenous).

Currently, both bio-identical estradiol and bio-identical progesterone are available in branded and generic USFDA (United States Food and Drug Administration) approved versions. A high demand for HT-containing bio-identical hormones exists in the market, which is shown by the estimated annual prescriptions of up to 21 million products containing natural progesterone, representing the most prescribed form of HT in the United States. Until recently, no USFDA approved product existed on the market comprising a combination of a bio-identical estradiol and a bio-identical progesterone. Because there was no single USFDA-approved formulation containing bio-identical estradiol and bio-identical progesterone before 2018, most of these (up to 18 million prescriptions) were for non-USFDA-approved compounded HT.

While combining both estradiol and progesterone in a single dosage form may be considered ideal for therapeutic reasons and convenient for patients, the difference in chemical structure between the compounds and their poor aqueous solubility present challenges in producing formulations with the appropriate bioavailability. Yet, many compounding pharmacies manufacture such combination products without the quality checks required of USFDA-approved drugs. Compounding pharmacies are monitored primarily by local state pharmacy boards with the USFDA having limited oversight. Some compounded drugs, including those used for HT, have been suspected of contamination and potency issues. USFDA surveys conducted in 2001 and 2006, to examine the identity, strength, quality, and purity of compounding products, found that 33% of the compounded products tested failed mostly due to superpotency or sub-potency (measured concentration was more than 10% from expected concentration), with potency ranging from 67.5% to 268.4%. This deviation from targeted drug concentrations in compounded products is in stark contrast to the typical failure rate of <2% seen in routine USFDA testing of commercially manufactured products. Thus, products from compounding pharmacies are approximately 10 times more likely to deviate significantly from the stated dose.

Various combinations of estrogen (conjugated equine estrogens [CEEs], ethinyl estradiol, 17β-estradiol and synthetic progestogens (norethindrone acetate, drospirenone, norgestimate, medroxyprogesterone acetate [MPA]) products are currently available, including oral and transdermal products.

USFDA first approved a drug product in the form of oral soft-gelatin capsule containing bio-identical estradiol and bio-identical progesterone in October 2018, which is commercially available as BIJUVA® (TherapeuticsMD, Boca Raton, Fla.), and which is indicated for the treatment of moderate to severe vasomotor symptoms due to menopause in post-menopausal women. Each capsule of BIJUVA® contains estradiol hemihydrate, progesterone, medium chain mono and di-glycerides, medium chain triglycerides, ammonium hydroxide, ethanol, ethyl acetate, FD&C Red #40, gelatin, glycerine, hydrolyzed gelatin, isopropyl alcohol, lecithin, lauroyl polyoxyl-32 glyceride, polyethylene glycol, polyvinyl acetate phthalate, propylene glycol, purified water and titanium dioxide. The BIJUVA® drug product is herein after used as reference composition-1.

U.S. Pat. Nos. 8,633,178; 8,846,648; 8,846,649; 8,987,237; 8,993,548; 8,993,549; 9,006,222; 9,114,145; 9,114,146; 9,301,920; 10,052,386 and 10,206,932 describe pharmaceutical compositions comprising progesterone, estradiol and a solubilizing agent, wherein the solubilizing agent comprises medium chain oil (C6-C12 oil).

Developing oral formulations of 17β-estradiol and progesterone in a single dosage form has always been challenging because of their differences in structure and solubility. There exists a need for developing a stable pharmaceutical composition for oral administration comprising 17β-estradiol and progesterone having an improved solubility, wherein estradiol does not precipitate for at least 12 months when stored at room temperature. There also exists a need for developing a stable pharmaceutical composition for oral administration comprising 17β-estradiol and progesterone having an improved stability, wherein the composition is stable for at least 12 months when stored at room temperature. There is also a need for developing an improved process for preparing a stable pharmaceutical composition for oral administration comprising 17β-estradiol and progesterone.

One other therapy involves administration of low dosages of one or more estrogen(s) or one or more chemical analogues. Another therapy involves administration of progesterone or one or more chemical analogues. In some patients administration of one or more estrogen(s) or one or more chemical analogues results in severe undesirable side effects such as endometrial hyperplasia (thickening) and endometrial cancer. Among other effects, progesterone administration acts to mitigate certain undesirable side effects from estradiol administration or naturally-occurring elevated blood levels including endometrial hyperplasia (thickening) and prevention or inhibition of endometrial cancer.

Progesterone is a C-21 steroidal sex hormone involved in the female menstrual cycle, pregnancy (supports gestation) and embryogenesis of humans and other species. Progesterone belongs to a class of hormones called progestogens, and is the major naturally occurring human progestogen. Like other steroids, progesterone consists of four interconnected cyclic hydrocarbons. Progesterone is hydrophobic, having a reported aqueous solubility of 0.007±0.0 mg/mL. Progesterone is poorly absorbed when administered orally.

Progesterone is currently marketed under the brand name PROMETRIUM® for use in the prevention of endometrial hyperplasia in non-hysterectomized postmenopausal women who are receiving conjugated estrogens tablets and also for use in secondary amenorrhea. PROMETRIUM® is currently available in the form of immediate-release soft-gelatin capsules (100 mg and 200 mg strengths) for oral administration. Each PROMETRIUM® capsule contains progesterone, peanut oil, gelatin, glycerin, lecithin, titanium dioxide, D&C Yellow No. 10, and FD&C Red No. 40. The PROMETRIUM® drug product is herein after used as reference composition-2. Progesterone in reference composition-2 is in the form of micronized progesterone, but with relatively large particle size fraction. Clinical trials involving postmenopausal women who were administered PROMETRIUM® once a day for five days resulted in the mean pharmacokinetic parameters listed in the table below, as reported in the package insert for PROMETRIUM®:

Pharmacokinetic parameters of PROMETRIUM ® capsules.

| | PROMETRIUM ® capsules daily dose | | |
|---|---|---|---|
| Parameters | 100 mg | 200 mg | 300 mg |
| Cₘₐₓ (ng/mL) | 17.3 ± 21.9 | 38.1 ± 37.8 | 60.6 ± 72.5 |
| Tₘₐₓ (hr) | 1.5 ± 0.8 | 2.3 ± 1.4 | 1.7 ± 0.6 |
| AUC₀₋₁₀ (ng*hr/mL) | 43.3 ± 30.8 | 101.2 ± 66.0 | 175.7 ± 170.3 |

The unusually high variability in the Cₘₐₓ and AUC, as evidenced by the large reported standard deviation, indicates that a significant percentage of patients are overdosed or receive a sub-optimal dose.

Progesterone when orally administered alone, or in combination with estradiol, has low bioavailability, largely due to poor water solubility. Therefore, in order to achieve therapeutic effective plasma concentration, high doses of progesterone have to be administered. There exists a need for developing a stable pharmaceutical composition suitable for oral administration comprising progesterone having an improved solubility and increased bioavailability of progesterone when compared with reference composition-2, wherein the composition is stable for at least 12 months when stored at room temperature.

There also exists a need for administering reduced daily doses of progesterone as compared to the dose of reference formulation, wherein the compositions exhibit increased bioavailability.

### SUMMARY OF THE INVENTION

In one aspect, the present application relates to a stable pharmaceutical composition suitable for oral administration to a subject in need thereof, comprising:
progesterone or a pharmaceutically acceptable salt thereof;
optionally, estradiol or a pharmaceutically acceptable salt thereof;
a solubilizing agent comprising long-chain oil;
a surfactant;
optionally, an antioxidant; and
optionally a co-solvent.

In another aspect, the present application relates to a stable pharmaceutical composition suitable for oral administration to a subject in need thereof, comprising:
progesterone or a pharmaceutically acceptable salt thereof;
estradiol or a pharmaceutically acceptable salt thereof;
a solubilizing agent comprising long-chain oil;
a surfactant;
optionally, an antioxidant; and
optionally a co-solvent.
   wherein the pharmaceutical composition upon oral administration exhibits bioequivalence to reference composition-1.

In another aspect, the present application relates to a stable pharmaceutical composition suitable for oral administration to a subject in need thereof, comprising:
progesterone or a pharmaceutically acceptable salt thereof;
estradiol or a pharmaceutically acceptable salt thereof;
a solubilizing agent comprising long-chain oil;
a surfactant;
optionally, an antioxidant; and
optionally a co-solvent.
   wherein the pharmaceutical composition upon oral administration exhibits increased bioavailability of progesterone when compared with reference composition-1.

In another aspect, the present application relates to a stable pharmaceutical composition suitable for oral administration to a subject in need thereof, comprising:
progesterone or a pharmaceutically acceptable salt thereof;
a solubilizing agent comprising a long-chain oil;
a surfactant;
optionally, an antioxidant; and
optionally a co-solvent.
   wherein the pharmaceutical composition upon oral administration exhibits increased bioavailability when compared with reference composition-2.

Another aspect relates to methods of treatment using the pharmaceutical compositions, e.g., orally administering an effective amount of the pharmaceutical compositions. Specifically provided is a method for treating vasomotor symptoms (VMS) related to menopause in a human patient. Also described is a method for preventing endometrial hyperplasia in non-hysterectomized postmenopausal women who are receiving conjugated estrogen. Further described is a method for treating secondary amenorrhea in a human patient.

Each aspect above may further have one or more of the following additional elements in any combination:

Element 1: wherein the long-chain oil comprises at least one of C₁₄-C₂₄ fatty acid mono, di or tri-esters of glycerol or mixtures thereof.

Element 2: wherein the long-chain oil comprises at least one of C₁₆-C₁₈ fatty acid mono, di or tri-esters of glycerol or mixtures thereof.

Element 3: wherein an average HLB value of the surfactant(s) is from about 9 to about 20.

Element 4: wherein the solubilizing agent is selected from the group consisting of a glyceryl mono-myristate, a glyceryl mono-palmitate, a glyceryl mono-oleate, a glyceryl dioleate, a glyceryl dioleate, a glyceryl mono-linoleate, a glycerol mono-stearate, a glyceryl palmitic/stearic, a glyceryl mono-α-linolenic acid, a glyceryl mono-elaidate, a glyceryl mono-vaccenate, a glyceryl mono-linoelaidate, a glyceryl mono-arachidonate, a glyceryl mono-eicosapentaenoate, a glyceryl mono-erucic acid, a glyceryl mono-docosahexaenoic acid, and mixtures thereof.

Element 5: wherein the solubilizing agent and surfactant are present in the composition in a weight ratio of about 50:50 to about 99:1, preferably about 60:40 to about 99:1.

Element 6: wherein the co-solvent is selected from the group consisting of ethanol, polyethylene glycol, propylene glycol, and mixtures thereof.

Element 7: wherein the co-solvent is ethanol.

Element 8: wherein the progesterone or a pharmaceutically acceptable salt thereof is a sole active ingredient.

Element 9: wherein the progesterone or a pharmaceutically acceptable salt thereof, and the estradiol or a pharmaceutically acceptable salt thereof, are both present as active ingredients.

Element 10: wherein the progesterone or a pharmaceutically acceptable salt thereof is present in an amount from about 30 mg to about 150 mg.

Element 11: wherein the composition provides increased progesterone bioavailability compared with reference composition-1 in a fed state or a fasted state.

Element 12: wherein the composition provides increased progesterone bioavailability compared to reference composition-2 in a fed state or a fasted state.

Element 13: wherein when the composition is stored for 6 months at 40° C./75% relative humidity (RH), the level of Impurity-M in the composition is less than about 0.2% (w/w) as measured by HPLC.

Element 14: wherein the co-solvent is present in an amount sufficient to inhibit phase separation for at least 24 hours when stored at 25±2° C. and 60±5% relative humidity (RH).

Element 15: wherein not less than 80% of the progesterone is released after about 45 minutes as determined using USP Apparatus III at 15 dpm in 3% SLS in 0.1 N HCl dissolution media at 37° C.

By way of non-limiting example, exemplary combinations applicable to the embodiments described in this application may include any combination with one or more of Elements 1-15, described above.

### BRIEF DESCRIPTION OF THE DRAWINGS

FIG. 1 illustrates the dissolution profile (% release) of estradiol release from BIJUVA®, Composition 30 and 31 (from Example 12) in dissolution media (3% SDS in 0.1N HCl) at 15 dips per minute (dpm).

FIG. 2 illustrates the dissolution profile (% release) of progesterone release of BIJUVA®, Composition 30 and 31 (from Example 12) in dissolution media (3% SDS in 0.1N HCl) at 15 dips per minute (dpm).

FIG. 3 illustrates amount of soluble progesterone in an in-vitro lipid digestion study of BIJUVA®, Composition 30, 31 and 34 (from Example 15).

### DETAILED DESCRIPTION OF THE INVENTION

Unless defined otherwise, all the technical and scientific terms used herein have the same meanings as commonly known by a person skilled in the art. In the case that there is a plurality of definitions for the terms herein, the definitions provided herein will prevail.

Unless specified otherwise, all the percentages, portions and ratios in the present invention are on weight basis.

Unless otherwise indicated, all numbers expressing quantities of ingredients, properties such as molecular weight, reaction conditions, and so forth used in the present specification and associated claims are to be understood as being modified in all instances by the term “about.”

Unless indicated to the contrary, the numerical parameters set forth in the following specification and attached claims are approximations that may vary depending upon the desired properties sought to be obtained by the embodiments of the present invention. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values.

While compositions and methods are described herein in terms of “comprising” various components or steps, the compositions and methods can also “consist essentially of” or “consist of” the various components and steps.

The terms “about” and “approximate”, when used along with a numerical variable, generally means the value of the variable and all the values of the variable within an experimental error (e.g., 95% confidence interval for the mean) or within a specified value±10% or within a broader range.

As used herein, “a,” “an,” “the,” “at least one,” and “one or more” are used interchangeably. As used herein, the term “or” is generally employed in its usual sense including “and/or” unless the content clearly dictates otherwise. Moreover, the indefinite articles “a” or “an,” as used in the claims, are defined herein to mean one or more than one of the elements that it introduces.

The term “treatment” as used herein includes any treatment of a condition or disease in a subject, or particularly a human, and may include: (i) preventing the disease or condition from occurring in the subject which may be predisposed to the disease but has not yet been diagnosed as having it; (ii) inhibiting the disease or condition, i.e., arresting its development; relieving the disease or condition, i.e., causing regression of the condition; or (iii) ameliorating or relieving the conditions caused by the disease, i.e., symptoms of the disease. “Treatment,” as used herein, could be used in combination with other standard therapies or alone.

The term “effective amount” refers to that amount which is sufficient to effect treatment, as defined herein, when administered to a subject in need of such treatment. The effective amount will vary depending on the subject and disease state being treated, the severity of the affliction and the manner of administration, and may be determined routinely by one of ordinary skill in the art.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

A “dosage,” “dosage form,” “dose unit” or “dose” as used herein means the amount of a pharmaceutical formulation comprising therapeutically active agent(s) administered at a time. “Dosage,” “dosage form,” “dose unit” or “dose” includes administration of one or more units of pharmaceutical formulation administered at the same time.

The terms “composition” and “formulation” refer to a pharmaceutical composition administered to a patient in need of treatment, which is typically in the form of a tablet, hard gelatin capsule, soft gelatin capsule, solution, suspension, emulsion and like.

An “active pharmaceutical ingredient” (API), as used herein, means the active compound or compounds used in formulating a drug product. APIs are generally safe for administering to animals, especially mammals, including humans, according to established governmental standards, including those promulgated by the USFDA.

The term “estrogen” refers to a group of several female sex hormones produced primarily by the ovaries, including estradiol, estrone, and estriol. As used herein, unless otherwise specified, estrogen refers to estradiol.

The term “estradiol” refers to (17beta)-estra-1,3,5(10)-triene-3,17-diol. Estradiol is also interchangeably called 17beta-estradiol, oestradiol, or E2, and is found endogenously in the human body. As used herein, estradiol refers to the bio-identical or body-identical form of estradiol found in the human body.

As used herein the term “progesterone” refers to progesterone free base or a pharmaceutically acceptable salt, solvate, anhydrous, hemihydrate, hydrate, co-crystal or polymorph thereof. As used herein, progesterone refers to the bio-identical or body-identical form of progesterone found in the human body.

The term “micronized progesterone”, as used herein, includes micronized progesterone having an D99 particle size value below about 45 microns, further below about 25 microns.

The term “stable” refers to both the physical and chemical stability of a composition in any form, such as a suspension. A composition is said to be stable if it exhibits minimal change over time relative to when it is manufactured. Stability is measured at various time points through a planned product expiration date with evaluation criteria including such items as appearance, phase separation between solubilizing agent and surfactant, pH of composition, content of active ingredient(s), and levels of degradation products, impurities, or related substances.

The term “shelf life” means the period beginning from manufacture of a formulation beyond which the formulation cannot be expected beyond reasonable doubt to yield the therapeutic outcome approved by a government regulatory agency.

As used herein, the term “storage” refers to the holding of a composition under controlled or uncontrolled conditions for a period ranging from a few minutes to several months or longer. Storage conditions that can be controlled include, for example, temperature, humidity, and the level of light. In many cases, storage of a pharmaceutical formulation is under industry acceptable standards and/or standards that are mandated by regulatory agencies, such as USFDA.

The term “oil” as used herein may be any pharmaceutically acceptable substance that would suspend and/or solubilize any suitable active ingredients as described herein. More specifically, oils may include, for example and without limitation, long-chain fatty acids, generally of the group known as long-chain fatty acids consisting of at least one mono-, di-, and triglyceride, or derivatives thereof, or combinations thereof.

Oils high in glycerides of polyunsaturated fatty acids are effective for purposes of the present invention. Such polyunsaturated fatty acids include linoleic, linolenic, santalbic, eleosoteric, punicic, trichosanic, and parinaric acids. Of the foregoing acids, linoleic and linolenic acids are particularly common in certain oils. Such oils include, for example, safflower oil, linseed oil, soybean oil, corn oil, and sunflower oil. Mixtures of these and other vegetable oils having similar properties can also be employed.

The term “long-chain” is used to describe the aliphatic chain length of fatty acid containing molecules. The term “long-chain” as used herein means any long-chain carbon-containing substance, including C₁₄-C₂₄ fatty acid esters of glycerol, fatty acids, and mono-, di-, and tri-glycerides of such substances.

The term “pharmaceutically acceptable excipient” as used herein means a diluent, carrier, or composition auxiliary, which is non-toxic and inert, which does not have undesirable effects on a subject to whom it is administered and is suitable for delivering a therapeutically active agent to the target site without affecting the therapeutic activity of the said active agent.

The term “solubilizing agent” refers to an agent or combination of agents that solubilize an active pharmaceutical ingredient (e.g., estradiol and/or progesterone). For example, and without limitation, suitable solubilizing agents include long-chain oils and other solvents and co-solvents that solubilize or dissolve an active pharmaceutical ingredient to a desirable extent. Solubilizing agents suitable for use in the formulations disclosed herein are pharmaceutical grade solubilizing agents (e.g., pharmaceutical grade long-chain oils). It will be understood by those of skill in the art that other excipients or components can be added to or mixed with the solubilizing agent to enhance the properties or performance of the solubilizing agent or resulting formulation. Examples of such excipients include, but are not limited to, surfactants, emulsifiers, thickeners, colorants, flavouring agents, etc. In some embodiments, the solubilizing agent is a long-chain oil and, in some other embodiments, the long-chain oil is combined with a co-solvent(s) or other excipient(s).

“Bioequivalence” refers to the absence of a significant difference between the bioavailability, i.e., the mean ratio of AUC (over 24 hours) and the mean ratio of C max is within 80% to 125% between two pharmaceutical drug products (e.g., a test composition and a reference composition) over the course of a period of time, at the same dose and under the same conditions. The determination of whether or not a test composition is bioequivalent to a reference composition is determined by performing a study, referred to as a bioequivalence or comparative bioavailability study, in a group of subjects under controlled conditions.

The term “bioavailability,” as used herein means the concentration of an active ingredient (e.g., progesterone or estradiol or estrone) in the blood (serum or plasma). The relative bioavailability may be measured as the concentration in the blood (serum or plasma) versus time. Other pharmacokinetic (pK) indicators may be used to measure and assess bioavailability, determined by suitable metrics including AUC, Cₘₐₓ and/or optionally, Tₘₐₓ.

In certain aspects, the bioavailability of the active ingredient(s) when formulated as described herein is at least about 15%, but may be greater than 20%, 25%, 30%, 35%, 40%, 45%, or 50% of the dose administered. As used herein, the term “increased bioavailability” refers to the increase in concentration of a drug in the body fluid provided by the compositions of the present invention over a reference drug product. Advantageously, the pharmaceutical compositions may have increased bioavailability compared to a commercially available reference product, thus requiring administration of a lower dosage amount. Thus, in certain aspects, the present application relates to improved pharmaceutical compositions, which exhibit increased bioavailability (e.g., higher AUC, Cₘₐₓ and/or Tₘₐₓ) compared to an existing commercial product, such as the existing formulations containing progesterone alone or in combination with estradiol. The BIJUVA® drug product marketed by TherapeuticsMD Inc. (approved under NDA No. 210132) is referred to as “reference composition-1” in the claims, and contains progesterone in combination with estradiol as the active ingredient. The PROMETRIUM® drug product marketed by Virtus Pharms (approved under NDA No. 020843) is referred to as “reference composition-2” in the claims, and contains progesterone as the active ingredient.

The term “AUC” as used herein, refers to the area under the curve that represents changes in blood concentration of progesterone, estradiol or estrone over time.

The term, “Cₘₐₓ” as used herein, refers to the maximum value of blood concentration shown on the curve that represents changes in blood concentrations of progesterone, estradiol or estrone over time.

The term, “Tₘₐₓ” as used herein, refers to the time that it takes for progesterone, estradiol or estrone blood concentration to reach the maximum value.

Collectively AUC, Cₘₐₓ, and, optionally, Tₘₐₓ are the principle pharmacokinetic parameters that can characterize the pharmacokinetic responses of a particular drug product such as progesterone in an animal or human subject.

Reference throughout this specification will be made to the administration of a pharmaceutical composition under fed or fasted conditions. It is well understood in the art that the pharmacokinetic performance of some compositions is affected by the presence or absence of food in the gastro-intestinal system, which is referred to in the art as “fed” or “fasted” states.

For example, the term “fasted state” means that the human or other mammal has not ingested 500 calories or more than 500 calories for at least two hours before taking the pharmaceutical composition and for at least two hours after taking the pharmaceutical composition.

As used herein, the term “fed state” refers to a human or other mammal who has eaten within about 30 minutes prior to drug administration. For example, the human or other mammal may have consumed at least 350 calories, or consumed an United States Food and Drug Administration (FDA) standard high fat breakfast (or other meal containing a comparable quantity of fat and calories) within said time period, which is high in both fat (approximately 50% of total calorie content of the meal) and calories (approximately 800-1000 calories) within about 30 minutes prior to drug administration.

In an embodiment, pharmaceutical compositions comprising estradiol, progesterone, a solubilizing agent, a surfactant and optionally a co-solvent, wherein said composition upon oral administration in fed state or fasted state exhibits bioequivalence to a commercially available BIJUVA®, and wherein said bioequivalence is established by at least one parameter that is selected from (i) a confidence interval for mean AUC₀₋ₜ between about 80% and about 125%; (ii) a confidence interval for mean AUC_{0-infinity} between about 80% and about 125%; (iii) a confidence interval for mean Cₘₐₓ between about 80% and about 125%; or (v) combinations thereof. Preferably, bioequivalence is established by at least one parameter that is selected from (i) a confidence interval for mean AUC₀₋ₜ between about 80% and about 125%; (ii) a confidence interval for mean AUC_{0-infinity} between about 80% and about 125%; (iii) a confidence interval for mean Cₘₐₓ between about 80% and about 125%.

In certain embodiments, the pharmaceutical compositions will preferably have an in vitro dissolution rate wherein not less than 80% of the progesterone is released after about 45 minutes. In other aspects, the in vitro dissolution rate not less than about 80% in 60 minutes. The in vitro dissolution rate may be determined using an USP Apparatus III at 15 dpm (dips per minute), in 3% SLS in 0.1 N HCl dissolution media at 37° C. In certain aspects, the in vitro release rate is chosen such that the peak plasma levels of one or both of the active ingredient(s) obtained in vivo occurs between about 1 and about 6 hours after administration of the composition to a patient.

The term “dispersed” as used herein refers to all means of establishing the presence of a beneficial agent in a composition according to the invention and includes a dispersion or suspension. The term “dispersion” refers to finely divided particles distributed in a solubilizing agent. In general, the particulate (dispersed) phase and the carrier medium (continuous phase) may be solids, liquids, or gaseous, but unless stated differently or otherwise clear from the context of the discussion, dispersion as used herein refers to solid particles, specifically micronized progesterone dispersed in a solubilizing agent.

There is a risk of estradiol and progesterone precipitation from compositions prepared with medium chain oil (C6-C12 oil), which hinders the absorption of estradiol and progesterone in the gastrointestinal tract. Long-chain oils (C14-C24 oils) help in absorption of estradiol and progesterone by providing microenvironment for estradiol and progesterone to remain in solubilized state without precipitation. It is challenging to develop pharmaceutical compositions comprising estradiol and progesterone using long-chain oil as solubilizing agent and still be bioequivalent to commercially available estradiol and progesterone combination product (such as BIJUVA®). Thus, there was a need to develop stable pharmaceutical compositions comprising estradiol and progesterone, which is addressed using long-chain oil as solubilizing agent, wherein said composition, upon oral administration exhibits bioequivalence to commercially available estradiol and progesterone combination product (such as BIJUVA®).

An embodiment of the present application relates to a stable pharmaceutical composition suitable for oral administration to a subject in need thereof, comprising:
progesterone or a pharmaceutically acceptable salt thereof;
estradiol or a pharmaceutically acceptable salt thereof;
a solubilizing agent comprising long-chain oil;
a surfactant;
optionally, an antioxidant; and
optionally a co-solvent.
   wherein the pharmaceutical composition upon oral administration exhibits bioequivalence to reference composition-1.

Another embodiment of the present application relates to a stable pharmaceutical composition suitable for oral administration to a subject in need thereof, comprising:
progesterone or a pharmaceutically acceptable salt thereof;
estradiol or a pharmaceutically acceptable salt thereof;
a solubilizing agent comprising a long-chain oil;
a surfactant;
optionally, an antioxidant; and
optionally a co-solvent.
   wherein the pharmaceutical composition upon oral administration exhibits increased bioavailability of progesterone when compared with reference composition-1.

An embodiment of the present application relates to a stable pharmaceutical composition suitable for oral administration to a subject in need thereof, comprising:
a progesterone or a pharmaceutically acceptable salt thereof;
a solubilizing agent comprising a long-chain oil;
a surfactant;
optionally, an antioxidant; and
optionally a co-solvent.
   wherein the pharmaceutical composition upon oral administration exhibits increased bioavailability of progesterone when compared with reference composition-2.

An embodiment of the present invention relates to stable pharmaceutical compositions for oral administration comprising estradiol and progesterone, wherein the composition is stable for at least 12 months when stored at room temperature.

Another embodiment of the present invention relates to stable pharmaceutical compositions for oral administration comprising estradiol and progesterone, wherein the composition further comprises a solubilizing agent and a surfactant.

Another embodiment relates to stable oral compositions comprising estrogen and progesterone, wherein the composition further comprises a solubilizing agent and a surfactant, and wherein solubilizing agent and surfactant are present in the composition in a weight ratio of 50:50 to 99:1.

Yet another embodiment of the present invention relates to stable compositions for oral administration comprising estradiol and progesterone, wherein the composition further comprises a solubilizing agent and a surfactant, and optionally other pharmaceutical acceptable excipients.

An aspect of the invention relates to stable compositions for oral administration, wherein the composition comprises solubilized estradiol, suspended progesterone, and a solubilizing agent, wherein the solubilizing agent is a long-chain (C₁₄-C₂₄) oil.

An aspect of the present invention relates to stable compositions of estradiol and progesterone for oral administration, wherein the composition further comprises a solubilizing agent and a surfactant, and wherein at least 90% of estradiol exists in a solubilized state and at least 75% of progesterone exists in a suspended state.

Another aspect of the present invention relates to stable compositions of estradiol and progesterone for oral administration having an improved solubility, wherein the composition further comprises a solubilizing agent and a surfactant, wherein estradiol does not precipitate for at least 12 months when stored at room temperature.

An aspect of the present invention relates to stable pharmaceutical compositions comprising 1 mg of estradiol and 100 mg of progesterone for oral administration, wherein the composition further comprises a solubilizing agent and a surfactant, and wherein at least 90% of estradiol exists in a solubilized state and at least 75% of progesterone exists in a suspended state.

An embodiment of the present invention relates to stable pharmaceutical compositions for oral administration comprising estradiol and progesterone, wherein the composition is stable for at least 12 months when stored at room temperature.

### Stability

As used herein, the term “stable” is defined as no more than (NMT) about 2% loss of progesterone and NMT about 4% of estradiol under typical commercial storage conditions. In certain embodiments, the compositions of the present invention will have NMT about 2% loss of progesterone and NMT about 4% of estradiol, under typical commercial storage conditions. The composition retains at least about 98% and 96% of the potency of progesterone and estradiol respectively after storing the composition at 40° C. and 75% relative humidity for at least three months. In certain aspects, the term “stable” refers to chemical stability, wherein NMT 2% w/w and NMT 4% of total related substances are formed for progesterone and estradiol respectively on storage at accelerated conditions of stability at 40° C. and 75% relative humidity or at 25° C. and 60% relative humidity or 2-8° C. for a period of at least six months or to the extent necessary for use of the composition.

In particular, the Impurity-M (i.e., (17α)-pregn-4-ene-3,20-dione) a degradant impurity of progesterone may be monitored. The structure of Impurity-M is shown below:

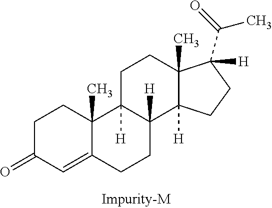

It was reported in a non-clinical review document submitted to the FDA by TherapeuticsMD that Impurity-M was observed above the ICH identification limit of 0.2% on accelerated stability conditions of Phase 3 clinical batches (see page 16 of Non-Clinical Review document of BIJUVA®).

Surprisingly it was found that a pharmaceutical composition comprising progesterone as a sole active ingredient or in combination with estradiol, long-chain oil and surfactant inhibits degradation of progesterone to form Impurity-M when stored for at least 6 months at 40° C./75% relative humidity (RH) condition exhibits less than about 0.2% (w/w) of Impurity-M as measured by HPLC.

In yet another embodiment, the invention relates to stable pharmaceutical composition comprising progesterone as sole active ingredient or in combination with estradiol, long-chain oil and surfactant, wherein the composition when stored for at least 6 months at 40° C./75% relative humidity (RH) condition exhibits less than about 0.2% (w/w) of Impurity-M as measured by HPLC.

In some embodiments, the composition comprises estradiol at a dosage of about 0.05, 0.1, 0.125, 0.15, 0.20, 0.25, 0.30, 0.35, 0.375, 0.40, 0.45, 0.50, 0.55, 0.60, 0.625, 0.65, 0.70, 0.75, 0.80, 0.85, 0.90, 0.95, 1.00, 1.125, 1.25, 1.375, 1.50, 1.625, 1.75, or 2.00 mg. In some embodiments, the composition comprises progesterone at a dosage of about 25, 50, 75, 100, 125, 150, 175, 200, 250, 300, 350, or 400 mg.

In some embodiments, estradiol is solubilized. Solubilized estradiol may include estradiol that is approximately 80% to 100% soluble in a solubilizing agent, including specifically embodiments that are: 80%, 81%, 82%, 83%, 84%, 85%, 86%, 87%, 88%, 89%, 90%, 91%, 92%, 93%, 94%, 95%, 96%, 97%, 98%, 99%, or 100% soluble in a solubilizing agent. Solubility may be expressed as a mass fraction (% w/w, also referred to as wt %). In some embodiments, estradiol is micronized. In some embodiments, micronized estradiol has an D50 particle size value of less than about 15 microns, less than about 10 microns, less than about 5 microns or less than about 3 microns. In some embodiments, micronized estradiol has an D90 particle size value of less than about 25 microns, less than about 20 microns, or less than about 15 microns. In some embodiments, the composition comprises micronized and partially solubilized estradiol.

In some embodiments, the micronized progesterone has a D99 particle size of about 5 μm to about 45 μm. In some embodiments, the micronized progesterone has a D99 particle size of about 5 μm to about 30 μm. In some embodiments, the micronized progesterone has a D99 particle size of about 5 μm to about 25 μm. In some embodiments, the micronized progesterone has a D99 particle size of about 5 μm to about 20 μm. In some embodiments, the micronized progesterone has a D99 particle size of about 5 μm to about 15 μm. In some embodiments, the micronized progesterone has a D99 particle size of about 5 μm to about 10 μm.

Estradiol and progesterone compositions of the present disclosure are prepared via blending with a solubilizing agent. In some embodiments, the solubilizing agent is a pharmaceutically acceptable oil that comprises a long-chain oil. In some embodiments, the solubilizing agent is a long-chain oil comprised substantially of C₁₄-C₂₄ long-chains, e.g., at least 20%, at least 30%, at least 40%, at least 50%, at least 60%, at least 70%, at least 80%, or at least 90% of the chains present in the oil are C₁₄-C₂₄. In some embodiments, the long-chain oil comprises at least one long-chain fatty acid such as long-chain fatty acids having at least one mono-, di-, or triglyceride, or derivatives thereof, or combinations thereof.

In an embodiment, the present invention provides a pharmaceutical composition comprising estradiol, progesterone, a solubilizing agent and a surfactant and optionally other pharmaceutical acceptable excipients, wherein solubilizing agent and surfactant are present in a weight ratio between 50:50 to 99:1. In some embodiments, solubilizing agent and surfactant are present in a weight ratio between 60:40 to 99:1. In some embodiments, solubilizing agent and surfactant are present in a weight ratio between 70:30 to 99:1. In some embodiments, solubilizing agent and surfactant are present in a weight ratio between 80:20 to 99:1. In some embodiments, solubilizing agent and surfactant are present in a weight ratio between 90:10 to 99:1. In some embodiments, the solubilizing agent and the surfactant are present in a weight ratio between 95:5 to 99:1.

In an embodiment, the present invention provides solubilizing agent selected from group consisting of long-chain fatty acid, long-chain fatty acid esters of glycerol, long-chain fatty acid esters of polyethylene glycol, long-chain fatty acid esters of propylene glycol or mixtures thereof.

In another embodiment, the present invention provides long-chain fatty acid selected from saturated long-chain fatty acid, polyunsaturated long-chain fatty acid and monounsaturated long-chain fatty acids or mixtures thereof.

In another embodiment, the present invention provides saturated long-chain fatty acid selected from group consisting of myristic acid, palmitic acid, stearic acid, arachidic acid, behenic acid, lignoceric acid, cerotic acid or mixtures thereof.

In another embodiment, the present invention provides unsaturated long-chain fatty acid selected from group consisting of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid or mixtures thereof.

In an embodiment, the present invention provides long-chain fatty acid esters of glycerol consisting of at least one mono-, di-, or triglyceride, or derivatives thereof, or combinations thereof, wherein long-chain fatty acid used to form glycerides is selected from group consisting of saturated long-chain fatty acid, unsaturated long-chain fatty acid or mixtures thereof.

In an embodiment, the present invention provides long-chain fatty acid esters of glycerol is selected from group consisting of at least one mono-, di-, or triglyceride, or derivatives thereof, or combinations thereof, wherein long-chain fatty acid used to form long-chain fatty acid esters of glycerol is selected from group consisting of myristic acid, palmitic acid, stearic acid, arachidic acid, behenic acid, lignoceric acid, cerotic acid, myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid.

In an embodiment, the present invention provides long-chain fatty acid esters of glycerol selected from the following table:

| Long-chain fatty acid esters | |
|---|---|
| of glycerol | Commercially available products |
| Glyceryl mono-myristate | Nikkol ® MGM |
| Glyceryl mono-palmitate | Emalex ® GMS-P |
| Glyceryl mono-oleate | Rylo ® series, Dimodan ® series, |
| | Emuldan ® 3-4, ALDO ® MO FG, |
| | Kessco ® GMO, Monomuls ® series, |
| | Tegino ®, Drewmulse ® GMO, Atlas ® |
| | G-695, GMOrphic ® 80, ADM DMG-40, |
| | 70, and 100, Peceol ®, Hodag ® |
| | GMO-D, Myverol ® |
| Glyceryl mono-, dioleate | Capmul ® GMO-K |
| Glyceryl dioleate | Capmul ® GDO |
| Glyceryl mono-linoleate | Maisine ®; Myverol ® |
| Glycerol mono-stearate | Capmul ® GMS, Myvaplex ®, |
| | Imwitor ® 191, Cutina ® GMS, Aldo ® |
| | MS, Nikkol ® MGS series) |
| Glyceryl palmitic/stearic | Cutina ® MD-A, Estagel ®-G18 |
| Glyceryl mono-α-linolenic | |
| acid | |
| Glyceryl mono-elaidate | |
| Glyceryl mono-vaccenate | |
| Glyceryl mono-linoelaidate | |
| Glyceryl mono-arachidonate | |
| Glyceryl mono- | |
| eicosapentaenoate | |
| Glyceryl mono-erucic acid | |
| Glyceryl mono- | |
| docosahexaenoic acid | |

In another embodiment, the long-chain fatty acid ester of glycerol is a monoglyceride, wherein long-chain fatty acid used to form long-chain fatty acid esters of glycerol is linoleic acid, α-linolenic acid or mixture thereof. In another embodiment, the long-chain fatty acid ester of glycerol is Maisine®CC.

In an embodiment, the present invention provides surfactant, which is selected from group consisting of polyethylene glycol (PEG)-long-chain fatty acid esters, PEG glycerol long-chain fatty acid esters, polyglycerol long-chain fatty acid esters, propylene glycol long-chain fatty acid esters, monoglycerides, diglycerides, polyethylene glycol sorbitan long-chain fatty acid esters, sugar esters, polyethylene glycol alkyl ethers, polyethylene glycol alkyl phenols, polyoxyethylene-polyoxypropylene block copolymers, sorbitan long-chain fatty acid esters, alcohol-oil trans-esterified oil surfactants or mixture thereof, wherein the long-chain fatty acid have a carbon chain length of from 014 to C24. In another embodiment, the surfactant is polyoxyethylene-polyoxypropylene block copolymers (poloxamers).

A large number of surfactants of different degrees of hydrophobicity or hydrophilicity can be prepared by reaction of alcohols or polyalcohols with a variety of natural and/or hydrogenated oils. Most commonly, the oils used are castor oil or hydrogenated castor oil, or an edible vegetable oil such as corn oil, olive oil, peanut oil, palm kernel oil, apricot kernel oil, or almond oil. In an embodiment, alcohols or poly-alcohols include glycerol, propylene glycol, ethylene glycol, polyethylene glycol, sorbitol, and pentaerythritol. Reaction between polyethylene glycol with oil results in polyethylene glycol-oil (PEG-oil) such as PEG 3-200 castor oil, PEG 5-100 hydrogenated castor oil, PEG 6-60 corn oil, PEG olive oil, PEG peanut oil, PEG hydrogenated palm kernel oil, PEG palm kernel oil, PEG apricot kernel oil, PEG triolein and PEG almond oil or mixture thereof.

In an embodiment, the PEG-oil is selected from group consisting of PEG-40 hydrogenated castor oil (Kolliphor® RH 40; HLB value lies between 14 and 16), PEG-32 stearate (GELUCIRE® 48/16; HLB value is 16), Stearoyl polyoxyl-32 glyceride (GELUCIRE® 50/13; HLB value is 13), PEG-25 trioleate (TAGAT® TO; HLB value is 11.3), PEG 60 corn glycerides (CROVOL® M70; HLB value is 15), PEG-60 almond oil (CROVOL® A70; HLB value is 10), PEG-40 palm kernel oil (CROVOL® PK70; HLB value>10), PEG-50 castor oil (EMALEX® C-50), PEG-50 hydrogenated castor oil (EMALEX® HC-50; HLB value is 14), PEG-5 hydrogenated castor oil, PEG-7 hydrogenated castor oil, PEG-9 hydrogenated castor oil, Linoleoyl Polyoxyl-6 glycerides (LABRAFIL® M 2125 CS; HLB value of 3 to 4), PEG-6 almond oil (LABRAFIL® M 1966 CS), Oleoyl polyoxyl-6 glycerides (LABRAFIL® M 1944 CS), PEG-6 olive oil (LABRAFIL® M 1980 CS), PEG-6 peanut oil (LABRAFIL® M 1969 CS), PEG-6 triolein (LABRAFIL® M 2735CS), PEG-8 corn oil (LABRAFIL® WL 2609 BS), PEG-20 corn glycerides (CROVOL® M40), and PEG-20 almond glycerides (CROVOL® A40), PEG-3 castor oil (Nikko CO-3), PEG-35 castor oil (Cremophor® EL), PEG-15 hydroxy stearate (SOLUTOL® HS 15). Preferably, the surfactant is selected from group consisting of GELUCIRE® 48/16, LABRAFIL® M 2125 CS, KOLLIPHOR® RH 40 or mixture thereof.

In yet another embodiment, the surfactant is polyglycerol long-chain fatty acid ester, especially, polyglyceryl-10 decaoleate (CAPROL® 10G10O).

In an embodiment, the present invention provides a pharmaceutical composition comprising one or more surfactant having HLB value between 9 to 16, which provides content uniformity of progesterone in unit dosage forms while manufacturing at commercial scale, enhances absorption of progesterone when orally administered.

The HLB is the ratio between the hydrophilic part and the lipophilic part in the molecule. The HLB (hydrophilic-lypophilic balance) value of the present invention can be a parameter used for determining whether a surfactant is hydrophilic or lipophilic, and takes a value from 0 to 20. The term HLB (“hydrophilic-lipophilic balance”) is well known to those skilled in the art, and denotes the hydrophilic-lipophilic balance of a surfactant.

It may be preferable that the average of the HLB value of the total surfactants in the composition according to the present invention range from about 9 to about 20.

In an embodiment, the present invention provides the average HLB value is determined by the weight average of the HLB values of all the surfactants, if two or more surfactants are used. For example, assuming that surfactants respectively having HLB values A, B and C are used, and that charged weights thereof in synthesis of the particle are respectively x, y and z, the weighted average value is calculated as follows: weight average value=(xA+yB+zC)+(x+y+z).

Hydrophilic surfactants having an HLB of 8-20 selected from group consisting of linoleoyl polyoxyl-6 glycerides (LABRAFIL® M 2125 CS), polyoxyethylene 20 sorbitan monooleate, polysorbate 80, sold under the trademark TWEEN® 80, available commercially from ICI; polyoxyethylene 20 sorbitan monolaurate (Polysorbate 20, TWEEN® 20); polyethylene (40 or 60) hydrogenated castor oil (available under the registered trademarks KOLLIPHOR® RH40 and RH60 from BASF); polyoxyethylene (35) castor oil (CREMOPHOR™ EL); polyethylene (60) hydrogenated castor oil (Nikkol™. HCO-60); alpha tocopheryl polyethylene glycol 1000 succinate (Vitamin E TPGS); glyceryl PEG 8 caprylate/caprate (available commercially under the registered trademark LABRASOL™ from Gattefosse); PEG 32 glyceryl laurate (sold commercially under the registered trademark GELUCIRE™ 44/14 by Gattefosse), polyoxyethylene fatty acid esters (available commercially under the registered trademark MYRJ from ICI), polyoxyethylene fatty acid ethers (available commercially under the registered trademark BRIJ from ICI). Preferred are Polysorbate 80, CREMOPHOR™ RH40 (BASF), and Vitamin E TPGS (Eastman). Most preferred are Polysorbate 80 and CREMOPHOR™ RH40.

The present invention provides co-solvents selected from the group consisting of diethylene glycol monoethyl ether (TRANSCUTOL® HP & TRANSCUTOL® P), ethanol, glycerine, polypropylene glycol, polyethylene glycol.

It was observed that when various pharmaceutical compositions for oral administration comprising progesterone in combination with or without estradiol, a solubilizing agent, a surfactant and optionally antioxidant were prepared, the compositions generally showed phase separation between solubilizing agent and surfactant in the composition. This physical instability further leads to lack of dose uniformity. Surprisingly, it was found that addition of co-solvent at weight percentage (total fill mass of the composition excluding capsule shell weight) of about 0.1% to about 10% in the composition is the amount of co-solvent in pharmaceutical composition is sufficient to inhibit phase separation for at least 24 hours when stored at 25±2° C. and 60±5% relative humidity (RH).

In an embodiment, the present invention provides a stable pharmaceutical composition comprising estradiol, progesterone, a long-chain oil, a surfactant, a co-solvent and optionally α-tocopherol, wherein co-solvent is in an amount sufficient to inhibit phase separation for at least 24 hours when stored at 25±2° C. and 60±5% relative humidity (RH). In certain embodiments, the pharmaceutical compositions the co-solvent is present in an amount sufficient to inhibit or avoid phase separation after 1 week, after 2 weeks, after 3 weeks, after 1 month, after 2 months, after 3 months, after 4 months, after 5 months, after 6 months, after 1 year, and after 2 years, when stored at 25±2° C. and 60±5% relative humidity (RH). By phase separation is meant that the composition does not show the creation of two distinct phases from a single homogeneous mixture, and may be determined by any suitable method used in the art, e.g., visual inspection.

In an embodiment, the present invention provides a stable pharmaceutical composition comprising estradiol, progesterone, a long-chain oil, a surfactant, a co-solvent and optionally α-tocopherol, wherein co-solvent is in an amount sufficient to inhibit phase separation for at least 24 hours when stored at 25±2° C. and 60±5% relative humidity (RH).

In an embodiment, the present invention provides a stable pharmaceutical composition comprising progesterone, a long-chain oil, a surfactant, a co-solvent and optionally α-tocopherol, wherein co-solvent is in an amount sufficient to inhibit phase separation for at least 24 hours when stored at 25±2° C. and 60±5% relative humidity (RH), wherein the progesterone is the sole active ingredient in the composition.

The present invention provides antioxidants selected from group consisting of α-tocopherol, β-carotene, butylated hydroxytoluene (BHT), butylated hydroxyanisole (BHA), tert-butylhydroquinone (TBHQ), propyl gallate (PG).

In an embodiment, the present invention provides a stable pharmaceutical composition comprising estradiol, progesterone, glyceryl mono-linoleate (Maisine® CC), polyethylene glycol monostearate (GELUCIRE® 48/16) and optionally ethanol, α-tocopherol.

In another embodiment, the present invention provides a stable pharmaceutical composition comprising estradiol, progesterone, glyceryl mono-linoleate (MAISINE® CC), Linoleoyl Polyoxyl-6 glycerides (LABRAFIL® M 2125 CS) and optionally ethanol and α-tocopherol.

In yet another embodiment, the present invention provides a stable pharmaceutical composition comprising estradiol, progesterone, glyceryl mono-linoleate (MAISINE® CC), polyoxyl-40 hydrogenated castor oil (KOLLIPHOR® RH 40) and optionally ethanol and α-tocopherol.

In yet another embodiment, the present invention provides a stable pharmaceutical composition comprising estradiol, progesterone, glyceryl mono-linoleate (MAISINE® CC), polyoxyl-40 hydrogenated castor oil (KOLLIPHOR® RH 40), ethanol and optionally α-tocopherol.

In yet another embodiment, the present invention provides a stable pharmaceutical composition comprising progesterone in combination with or without estradiol, glyceryl mono-linoleate (MAISINE® CC), polyoxyl-40 hydrogenated castor oil (KOLLIPHOR® RH 40), ethanol and optionally α-tocopherol.

In yet another embodiment, the present invention provides a stable pharmaceutical composition comprising progesterone, glyceryl mono-linoleate (MAISINE® CC), polyoxyl-40 hydrogenated castor oil (KOLLIPHOR® RH 40), ethanol and optionally α-tocopherol.

In another embodiment, the present invention provides a stable pharmaceutical composition comprising estradiol, progesterone, glyceryl mono-linoleate (MAISINE® CC), polyglyceryl-10 decaoleate (CAPROL® 10G100) and optionally ethanol and α-tocopherol.

The present invention also relates to an improved process for preparing a stable pharmaceutical composition comprising estradiol and progesterone for the treatment of moderate to severe vasomotor symptoms due to menopause in post-menopausal women.

Another aspect of the present invention also relates to an improved process for preparing stable compositions of estradiol and progesterone for oral administration, wherein the process involves heating the solubilizing agent up to 45° C., and then dissolving estradiol in the solubilizing agent.

Another aspect of the present invention also relates to an improved process for preparing stable compositions comprising estradiol and progesterone for oral administration, wherein the process involves heating the solubilizing agent up to 45° C., and then dissolving estradiol in the solubilizing agent.

### Methods of Treatment

The pharmaceutical compositions may be orally administered for treating vasomotor symptoms (VMS) related to menopause in a human patient, e.g., such as for administration to a woman with a uterus for the treatment of moderate to severe vasomotor symptoms due to menopause. For instance, the pharmaceutical compositions may be used to treat or reduce symptoms such as moderate to severe hot flashes, night sweats, and vaginal atrophy, and also to prevent osteoporosis.

In certain embodiments, the pharmaceutical compositions may be used for the prevention of endometrial hyperplasia in non-hysterectomized postmenopausal women who are receiving conjugated estrogens tablets. In this regard, taking estrogen alone is associated with an increased chance of endometrial hyperplasia, which may lead to cancer of the lining of the uterus (womb). Therefore, the addition of a progestin is recommended for a woman with a uterus to reduce the chance of getting cancer of the uterus. For example, a post-menopausal woman with a uterus who is taking estrogens may take a single daily dose of a pharmaceutical compositions as described here comprising 200 mg progesterone at bedtime for 12 continuous days per 28 day cycle. Preferably, due to the increased bioavailability of the pharmaceutical compositions as described here, the dosage will be less than 200 mg progesterone.

In certain embodiments, the pharmaceutical compositions may be used for the treatment of secondary amenorrhea. By “secondary amenorrhea” is meant the absence of menstrual periods due to causes not due to underlying disease, e.g., menopause, pregnancy, use of birth control, medication side effects, delayed puberty, stress, polycystic ovary syndrome, hypothalamic amenorrhea, hyperprolactinemia, or primary ovarian insufficiency. In certain cases, if a woman does not produce sufficient amounts of progesterone, menstrual irregularities can occur. Therefore, progesterone may be prescribed in such patients. For example, for the treatment of secondary amenorrhea, a single daily dose of a pharmaceutical compositions as described here comprising 400 mg progesterone at bedtime for 10 days may be administered. Preferably, due to the increased bioavailability of the pharmaceutical compositions as described here, the dosage will be less than 400 mg progesterone.

The subjects may be pre-menopausal, peri-menopausal, menopausal or post-menopausal.

### Dosage and Administration

Pharmaceutical compositions comprising estradiol and progesterone as described herein (e.g., compositions comprising solubilized estradiol, suspended progesterone, and a medium chain solubilizing agent) can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. Pharmaceutical compositions can be formulated for any appropriate manner of administration, including, for example, topical, oral, nasal, intrathecal, rectal, vaginal, sublingual or parenteral administration.

For preparing pharmaceutical compositions from the compounds of this disclosure, the pharmaceutically acceptable compositions can be either solid or liquid. Solid form preparations include powders, tablets, pills, capsules, cachets, suppositories, and dispersible granules. A solid preparation can comprise one or more substances, which may also act as diluents, flavouring agents, binders, preservatives, tablet disintegrating agents, or an encapsulating material. Details on techniques for formulation and administration are well described in the scientific and patent literature, see, e.g., Remington's Pharmaceutical Sciences, 22th Edition, Loyd V. Allen, Jr., Ed., Pharmaceutical Press, Easton, Pa. (2012) (“Remington's”).

In general, the type of composition is selected based on the mode of administration. A pharmaceutical composition (e.g., for oral administration or delivery by injection) can be in the form of a liquid (e.g., an elixir, syrup, solution, emulsion or suspension). Alternatively, a pharmaceutical composition as described herein can take the form of a pill, tablet, or capsule containing the liquid oil, and thus, the composition can contain any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof.

Administration of the compositions of this disclosure can be carried out via any of the accepted modes of administration for oral administration.

In some embodiments, a pharmaceutical composition as described herein is administered once daily fora period of 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 days or more. In some embodiments, a pharmaceutical composition as described herein is administered daily for at least one week, at least two weeks, at least three weeks, at least four weeks, at least one month, at least two months, at least three months, at least four months, at least five months, at least six months, at least seven months, at least eight months, at least nine months, at least ten months, at least eleven months, at least twelve months, or more. In some embodiments, a pharmaceutical composition as described herein is administered as a continuous-combined therapy regimen.

In some embodiments, a 28-day or monthly regimen of daily doses is packaged in a single kit (e.g., a blister pack) having administration days identified to improve compliance and reduce associated symptoms, among others. In some embodiments, each daily dose contains both estradiol and progesterone. In some embodiments, one or more of the daily doses contains no estradiol or no progesterone. Daily doses that comprise no estradiol or progesterone API may be referred to as placebos. A blister pack can have a plurality of scores or perforations separating the blister pack into 28 days. Each day may further comprise a single blister or a plurality of blisters. In various embodiments, each unit dose may contain micronized or partially solubilized, or fully solubilized progesterone or solubilized estradiol in amounts as set forth herein, although other dose ranges may be contemplated. In addition, kits having other configurations are also contemplated herein. For example, without limitation, kits having such blister packs may contain any number of daily doses.

In certain embodiments, an oral dosage form (e.g., one capsule) may be taken as a single dose at bedtime, or orally each evening with food.

Timing for dosage administration is often varied cyclically, with estrogens taken daily and progesterone taken for approximately two weeks of every month; a method often referred to as “Cyclic-Sequential” or “Sequentially-Combined” HRT. This method is intended to mimic the natural menstrual cycle and typically causes menstruation similar to a period after the progesterone is stopped. This regimen is most typically used in peri-menopausal or newly menopausal women as the alternative continuous method often results in irregular bleeding in such women. An alternate method, a constant dosage with both estrogen and progesterone taken daily, is called “continuous-combined HRT.” This method usually results in no menstruation and is used most often after a woman has been menopausal for some time.

### EXAMPLES

The following examples are exemplary and not intended to be limiting. The present disclosure provides many different embodiments for implementing the features of the invention, and the following examples describe certain embodiments. It will be appreciated that other modifications and methods known to one of ordinary skill in the art can also be applied to the following experimental procedures, without departing from the scope of the invention.

General High-Performance Liquid Chromatography (HPLC) Procedure

As explained in detail below, the following HPLC procedure can be used to detect and quantify amount of solubilized estradiol and progesterone. The materials and general conditions are listed below:

TABLE 1
Chromatographic conditions

| Chromatographic Mode | Gradient |
|---|---|
| Column | ACE C18, 150 × 4.6, 3μ |
| Wavelength | Progesterone-UV 254 nm and |
| | Estradiol FLD excitation: 282 nm, |
| | Emission: 316 nm |
| Flow rate | 1.0 mL/Min |
| Injection volume | 3 μL |
| Column temperature | 30° C. |
| Sample temperature | 25° C. |
| Retention time | Estradiol: about 4.5 minutes |
| | Progesterone: about 9.5 minutes |
| Run time | 25 minutes |
| Needle Wash | 100% Methanol |
| Mobile Phase A | 70:30 Methanol:Water (v/v) |
| Mobile Phase B | 100% Methanol |
| Blank | Methanol |

TABLE 2
Gradient Programme

| Time in Min | % Mobile Phase A | % Mobile Phase B |
|---|---|---|
| 0 | 100 | 0 |
| 10 | 100 | 0 |
| 12 | 0 | 100 |
| 18 | 0 | 100 |
| 19 | 100 | 0 |
| 25 | 100 | 0 |

### Standards Preparation:

Estradiol Stock: Weigh accurately 25 mg of estradiol working standard/reference standard into a 50 mL volumetric flask, add 30 mL methanol and sonicate to dissolve. Allow to cool to room temperature, make up to volume with methanol.

Standard Solution: Weigh accurately 80 mg of Progesterone working standard/reference standard into a 100 mL volumetric flask, add 60 mL methanol and sonicate to dissolve. Allow to cool to room temperature, add 10 mL above estradiol stock solution into it and make up to volume with methanol.

### Sample Preparation:

Capsules:

1. Take not less than 5 capsules and cut the capsules slowly from top edge and collect the fill content of capsule into a small volume beaker. Allow it to settle down the solid content at the bottom of the beaker. Take sufficient quantity of upper layer (liquid portion) of sample into a 2 mL microcentrifuge tube.

2. Centrifuge the sample at 5000 RPM about 10 mins. Ensure the clear separation of liquid portion form solid content.

3. Accurately weigh about 200 mg of sample (Liquid) into a 20 mL volumetric flask by using a dropper pipette. Add 15 mL of methanol and sonicate for 15 mins. Allow the sample to cool to room temperature and make up to volume with methanol.

4. Filter a portion of solution with 0.45p Nylon filter and collect into HPLC vial and analyse.

Bulk Sample:

1. Take sufficient quantity of upper layer (liquid portion) of sample into a 2 mL microcentrifuge tube from bulk quantity.

2. Centrifuge the sample at 5000 RPM about 10 mins. Ensure the clear separation of liquid portion form solid content.

3. Accurately weigh about 200 mg of sample (Liquid) into a 20 mL volumetric flask by using a dropper pipette. Add 15 mL of methanol and sonicate for 15 mins. Allow the sample to cool to room temperature and make up to volume with methanol.

4. Filter a portion of solution with 0.45p Nylon filter and collect into HPLC vial and analyse.

### Example 1

TABLE 3
Quantitative Formula: Batch Size 500 capsules

| | Composition 1 | | | Composition 2 | | |
|---|---|---|---|---|---|---|
| | mg/ | Qty/ | | mg/ | Qty/ | |
| Components | capsule | 150 g | | capsule | 150 g | |
| Micronized progesterone | 100.00 | 50 | g | 100.00 | 50 | g |
| Estradiol hemihydrate | 1.02 | 0.51 | g | 1.02 | 0.51 | g |
| Maisine ® CC | 194.65 | 97.33 | g | 190.67 | 95.34 | g |
| Gelucire ® 48/16 | 3.98 | 1.99 | g | 7.96 | 3.98 | g |
| Ethanol absolute | 0.00 | 0.00 | | 0.00 | 0.00 | g |
| α-tocopherol | 0.35 | 0.17 | g | 0.35 | 0.17 | g |
| Total fill weight | 300.00 | 150 | g | 300.00 | 150 | g |
| | Composition 3 | | | Composition 4 | | |
| | mg/ | Qty/ | | mg/ | Qty/ | |
| Components | capsule | 150 g | | capsule | 150 g | |
| Micronized progesterone | 100.00 | 50 | g | 100.00 | 50 | g |
| Estradiol hemihydrate | 1.02 | 0.51 | g | 1.02 | 0.51 | g |
| Maisine ® CC | 189.63 | 94.82 | g | 189.13 | 94.56 | g |
| Gelucire ® 48/16 | 9.00 | 4.5 | g | 8.50 | 4.25 | g |
| Ethanol absolute | 0.00 | 0.00 | | 1.00 | 0.5 | g |
| α-tocopherol | 0.35 | 0.17 | g | 0.35 | 0.17 | g |
| Total fill weight | 300.00 | 150 | g | 300.00 | 150 | g |

Composition 5

| Components | mg/capsule | Qty/150 g | |
|---|---|---|---|
| Micronized progesterone | 100 | 50 | g |
| Estradiol hemihydrate | 1.02 | 0.51 | g |
| Maisine ® CC | 190.98 | 95.49 | g |
| Gelucire ® 48/16 | 8 | 4 | g |
| Total fill weight | 300 | 150 | g |

Manufacturing Procedure of Compositions 1-4:

MAISINE® CC was heated to 40° C. and the temperature was maintained at 40° C. until a final suspension was obtained. GELUCIRE® 48/16 was added to MAISINE® CC and mixed until GELUCIRE® 48/16 was completely dissolved. Ethanol was added to the previous mix and stirred until clear solution was obtained. To the above clear solution, α-tocopherol was added and mixed until clear solution was obtained. Weighed quantity of micronized progesterone was added to clear solution and mixed until uniform dispersion was obtained and estradiol was added to uniform dispersion and mixed for 30 minutes to obtain final suspension. This final suspension was stored at room temperature until the encapsulation.

Manufacturing Procedure of Composition 5:

MAISINE® CC was heated to 40° C. and temperature was maintained at 40° C. until a final suspension was obtained. GELUCIRE® 48/16 was added to MAISINE® CC and mixed until GELUCIRE® 48/16 was completely dissolved. Weighed quantity of micronized progesterone was added to clear solution and mixed until uniform dispersion was obtained and estradiol was added to uniform dispersion and mixed for 30 minutes to obtain final suspension. This final suspension was stored at room temperature until the encapsulation.

### Example 2

TABLE 4
Quantitative Formula: Batch Size 500 capsules

| | Composition 6 | | | Composition 7 | | |
|---|---|---|---|---|---|---|
| | mg/ | Qty/ | | mg/ | Qty/ | |
| Components | capsule | 150 g | | capsule | 150 g | |
| Micronized progesterone | 100.00 | 50 | g | 100 | 50 | g |
| Estradiol hemihydrate | 1.02 | 0.51 | g | 1.02 | 0.51 | g |
| Maisine ® CC | 167.03 | 83.515 | | 183 | 91.5 | g |
| Gelucire ® 48/16 | 8.00 | 4.00 | | 8 | 4 | g |
| PEG 400 | 15.98 | 7.99 | | 0 | 0 | |
| Ethanol absolute | 7.99 | 3.995 | | 7.98 | 3.99 | g |
| Total fill weight | 300.00 | 150 | g | 300 | 150 | g |

Manufacturing Procedure:

MAISINE® CC was heated to 40° C. GELUCIRE® 48/16 was added to MAISINE® CC and mixed until dissolved completely. Ethanol and PEG 400 was added to the previous mix and stirred until clear solution obtained. Weighed quantity of micronized progesterone was added to clear solution and mixed until uniform dispersion was obtained and estradiol was added to uniform dispersion and mixed for 30 minutes to obtain final suspension. This final suspension was stored at room temperature until the encapsulation.

### Example 3

TABLE 5
Quantitative Formula: Batch Size 500 capsules

| | Composition 8 | | | Composition 9 | | |
|---|---|---|---|---|---|---|
| | mg/ | Qty/ | | mg/ | Qty/ | |
| Components | capsule | 150 g | | capsule | 150 g | |
| Micronized progesterone | 100.00 | 50 | g | 100 | 50 | g |
| Estradiol hemihydrate | 1.02 | 0.51 | g | 1.02 | 0.51 | g |
| Maisine ® CC | 193.63 | 96.81 | g | 188.68 | 94.34 | g |
| Labrafil ® M 2125 CS | 5 | 2.5 | g | 9.95 | 4.97 | g |
| Ethanol absolute | 0.00 | 0.00 | | 0.00 | 0.00 | |
| α-tocopherol | 0.35 | 0.17 | g | 0.35 | 0.17 | g |
| Total fill weight | 300 | 150 | g | 300 | 150 | g |
| | Composition 10 | | | Composition 11 | | |
| | mg/ | Qty/ | | mg/ | Qty/ | |
| Components | capsule | 150 g | | capsule | 150 g | |
| Micronized progesterone | 100.00 | 50 | g | 100.00 | 50 | g |
| Estradiol hemihydrate | 1.02 | 0.51 | g | 1.02 | 0.51 | g |
| Maisine ® CC | 178.73 | 89.36 | g | 158.63 | 79.32 | g |
| Labrafil ® M 2125 CS | 19.90 | 9.95 | g | 39 | 19.5 | g |
| Ethanol absolute | 0.00 | 0.00 | | 1.00 | 0.5 | g |
| α-tocopherol | 0.35 | 0.17 | g | 0.35 | 0.17 | g |
| Total fill weight | 300.00 | 150 | g | 300.00 | 150 | g |

Manufacturing Procedure:

MAISINE® CC was heated to 40° C. LABRAFIL® M 2125 CS was added to MAISINE® CC and mixed until dissolved completely. Ethanol was added to the previous mix and stirred until clear solution obtained. To the above clear solution, α-tocopherol was added and mixed until clear solution was obtained. Weighed quantity of micronized progesterone was added to clear solution and mixed until uniform dispersion was obtained and estradiol was added to uniform dispersion and mixed for 30 minutes to obtain final suspension. This final suspension was stored at room temperature until the encapsulation.

### Example 4

TABLE 6
Quantitative Formula: Batch Size 500 capsules

| | Composition 12 | | | Composition 13 | | |
|---|---|---|---|---|---|---|
| | mg/ | Qty/ | | mg/ | Qty/ | |
| Components | capsule | 150 g | | capsule | 150 g | |
| Micronized progesterone | 100.00 | 50 | g | 100.00 | 50 | g |
| Estradiol hemihydrate | 1.02 | 0.51 | g | 1.02 | 0.51 | g |
| Maisine ® CC | 190.00 | 95 | | 160.00 | 80 | g |
| Labrafil ® M 2125 CS | 9.00 | 4.5 | | 39 | 19.5 | g |
| Total fill weight | 300 | 150 | g | 300 | 150 | g |
| | Composition 14 | | | Composition 15 | | |
| | mg/ | Qty/ | | mg/ | Qty/ | |
| Components | capsule | 150 g | | capsule | 150 g | |
| Micronized progesterone | 100.00 | 50 | g | 100.00 | 50 | g |
| Estradiol hemihydrate | 1.02 | 0.51 | g | 1.02 | 0.51 | g |
| Maisine ® CC | 179.00 | 89.5 | | 194.00 | 97 | g |
| Labrafil ® M 2125 CS | 20.00 | 10 | | 5.00 | 2.5 | g |
| Total fill weight | 300.00 | 150 | g | 300.00 | 150 | g |

Composition 16

| Components | mg/capsule | Qty/150 g | |
|---|---|---|---|
| Micronized progesterone | 100.00 | 50 | g |
| Estradiol hemihydrate | 1.02 | 0.51 | g |
| Maisine ® CC | 160.00 | 80 | g |
| Labrafil ® M 2125 CS | 39.00 | 19.5 | g |
| Total fill weight | 300.00 | 150 | g |

Manufacturing Procedure:

MAISINE® CC was heated to 40° C. LABRAFIL® M 2125 CS was added to MAISINE® CC and mixed until dissolved completely to obtain clear solution. Weighed quantity of micronized progesterone was added to clear solution and mixed until uniform dispersion is obtained and estradiol was added to uniform dispersion and mixed for 30 minutes to obtain final suspension. This final suspension was stored at room temperature until the encapsulation.

### Example 5

TABLE 7
Quantitative Formula: Batch Size 500 capsules

| | Composition 17 | | | Composition 18 | | |
|---|---|---|---|---|---|---|
| | mg/ | Qty/ | | mg/ | Qty/ | |
| Components | capsule | 150 g | | capsule | 150 g | |
| Micronized progesterone | 100.00 | 50 | g | 100.00 | 50 | g |
| Estradiol hemihydrate | 1.02 | 0.51 | g | 1.02 | 0.51 | g |
| Maisine ® CC | 177.28 | 88.64 | g | 175.48 | 87.74 | g |
| Labrafil ® M 2125 CS | 19.7 | 9.85 | g | 19.5 | 9.75 | g |
| Ethanol absolute | 2 | 1 | g | 4 | 2 | g |
| Total fill weight | 300 | 150 | g | 300 | 150 | g |

Composition 19

| Components | mg/capsule | Qty/150 g | |
|---|---|---|---|
| Micronized progesterone | 100.00 | 50 | g |
| Estradiol hemihydrate | 1.02 | 0.51 | g |
| Maisine ® CC | 173.35 | 86.675 | g |
| Labrafil ® M 2125 CS | 19.63 | 9.815 | g |
| Ethanol absolute | 6 | 3 | g |
| Total fill weight | 300 | 150 | g |

Manufacturing Procedure:

MAISINE® CC was heated to 40° C. LABRAFIL® M 2125 CS was added to MAISINE® CC and mixed until dissolved completely to obtain clear solution. Ethanol was added to the previous mix and stirred until clear solution obtained. Weighed quantity of micronized progesterone was added to clear solution and mixed until uniform dispersion is obtained and estradiol was added to uniform dispersion and mixed for 30 minutes to obtain final suspension. This final suspension was stored at room temperature until the encapsulation.

### Example 6

TABLE 8
Quantitative Formula: Batch Size 500 capsules

| | Composition 20 | | | Composition 21 | | |
|---|---|---|---|---|---|---|
| | mg/ | Qty/ | | mg/ | Qty/ | |
| Components | capsule | 150 g | | capsule | 150 g | |
| Micronized progesterone | 100.00 | 50 | g | 100.00 | 50 | g |
| Estradiol hemihydrate | 1.02 | 0.51 | g | 1.02 | 0.51 | g |
| Maisine ® CC | 194.6 | 97.33 | g | 188.68 | 94.34 | g |
| Kolliphor ® RH-40 | 3.98 | 1.99 | g | 9.95 | 4.97 | g |
| Ethanol absolute | 0.00 | 0.00 | | 0.00 | 0.00 | |
| α-tocopherol | 0.35 | 0.17 | g | 0.35 | 0.17 | g |
| Total fill weight | 300.00 | 150 | g | 300.00 | 150 | g |
| | Composition 22 | | | Composition 23 | | |
| | mg/ | Qty/ | | mg/ | Qty/ | |
| Components | capsule | 150 g | | capsule | 150 g | |
| Micronized progesterone | 100.00 | 50 | g | 100.00 | 50 | g |
| Estradiol hemihydrate | 1.02 | 0.51 | g | 1.02 | 0.51 | g |
| Maisine ® CC | 119.22 | 59.61 | | 168.43 | 84.22 | g |
| Kolliphor ® RH-40 | 69.41 | 34.705 | | 29.20 | 14.6 | g |
| Ethanol absolute | 10.00 | 5 | | 1.00 | 0.5 | g |
| α-tocopherol | 0.35 | 0.175 | | 0.35 | 0.17 | g |
| Total fill weight | 300.00 | 150 | g | 300.00 | 150 | g |

Manufacturing Procedure:

MAISINE® CC was heated to 40° C. KOLLIPHOR® RH-40 was added to MAISINE® CC and mixed until dissolved completely. Weighed quantity ethanol was added to the previous mix and stirred until clear solution obtained. To the above clear solution, α-tocopherol was added and mixed until clear solution was obtained. Weighed quantity of micronized progesterone was added to clear solution and mixed until uniform dispersion was obtained and estradiol was added to uniform dispersion and mixed for 30 minutes to obtain final suspension. This final suspension was stored at room temperature until the encapsulation.

### Example 7

TABLE 9
Quantitative Formula: Batch Size 500 capsules

| | Composition 24 | | | Composition 25 | | |
|---|---|---|---|---|---|---|
| | mg/ | Qty/ | | mg/ | Qty/ | |
| Components | capsule | 150 g | | capsule | 150 g | |
| Micronized progesterone | 100.00 | 50 | g | 100.00 | 50 | g |
| Estradiol hemihydrate | 1.02 | 0.51 | g | 1.02 | 0.51 | g |
| Maisine ® CC | 99.33 | 49.65 | g | 109.33 | 54.67 | g |
| Kolliphor ® RH-40 | 79.65 | 39.82 | g | 79.65 | 39.82 | g |
| Ethanol absolute | 20 | 10 | g | 10 | 5 | g |
| Total fill weight | 300.00 | 150 | g | 300.00 | 150 | g |

Composition 26

| Components | mg/capsule | Qty/150 g | |
|---|---|---|---|
| Micronized progesterone | 100.00 | 50 | g |
| Estradiol hemihydrate | 1.02 | 0.51 | g |
| Maisine ® CC | 115.33 | 57.67 | g |
| Kolliphor ® RH-40 | 79.65 | 39.82 | g |
| Ethanol absolute | 4 | 2 | g |
| Total fill weight | 300.00 | 150 | g |

Manufacturing Procedure:

MAISINE® CC was heated to 40° C. KOLLIPHOR® RH-40 was added to MAISINE® CC and mixed until dissolved completely. Ethanol was added to the previous mix and stirred until clear solution obtained. Weighed quantity of micronized progesterone was added to clear solution and mixed until uniform dispersion was obtained and estradiol was added to uniform dispersion and mixed for 30 minutes to obtain final suspension. This final suspension was stored at room temperature until the encapsulation.

### Example 8

TABLE 10
Quantitative Formula: Batch Size 500 capsules

| | Composition 27 | | | Composition 28 | | |
|---|---|---|---|---|---|---|
| | mg/ | Qty/ | | mg/ | Qty/ | |
| Components | capsule | 150 g | | capsule | 150 g | |
| Micronized progesterone | 100.00 | 50 | g | 100.00 | 50 | g |
| Estradiol hemihydrate | 1.02 | 0.51 | g | 1.02 | 0.51 | g |
| Caprol ® 10G10O | 194.65 | 97.33 | g | 188.68 | 94.34 | g |
| Labrafil ® M 2125 CS | 3.98 | 1.99 | g | 9.95 | 4.97 | g |
| Ethanol absolute | 0.00 | 0.00 | | 0.00 | 0.00 | |
| α-tocopherol | 0.35 | 0.17 | g | 0.35 | 0.17 | g |
| Total fill weight | 300.00 | 150 | g | 300.00 | 150 | g |

Composition 29

| Components | mg/capsule | Qty/150 g | |
|---|---|---|---|
| Micronized progesterone | 100.00 | 50 | g |
| Estradiol hemihydrate | 1.02 | 0.51 | g |
| Caprol ® 10G10O | 178.23 | 89.12 | g |
| Labrafil ® M 2125 CS | 19.40 | 9.7 | g |
| Ethanol absolute | 1.00 | 0.5 | g |
| α-tocopherol | 0.35 | 0.17 | g |
| Total fill weight | 300.00 | 150 | g |

Manufacturing Procedure:

CAPROL® 10G100 was heated to 40° C. LABRAFIL® M 2125 CS was added to CAPROL® 10G10O and mixed until dissolved completely. Ethanol was added to the previous mix and stirred until clear solution obtained. To the above clear solution, α-tocopherol was added and mixed until clear solution was obtained. Weighed quantity of micronized progesterone was added to clear solution and mixed until uniform dispersion was obtained and estradiol was added to uniform dispersion and mixed for 30 minutes to obtain final suspension. This final suspension was stored at room temperature until the encapsulation.

### Example 9

Encapsulation:

The final suspension of compositions 1-29 were transferred to the soft gel machine intermediate hopper. The gel ribbons were fed on cooling drums lined up with the die rolls. Two plasticized gel ribbons were lubricated with a mix of lecithin and medium-chain triglycerides, and continuously and simultaneously fed with the blend formulation between the rollers of the rotary die mechanism where the capsule was simultaneously filled, shaped, hermetically sealed and cut from the gel ribbon. The sealing of the capsule was achieved by mechanical pressure on the die rolls and the heating of the ribbons by the wedge. Formed capsules were transferred into tumbler dryers where they will be pre-dried at room temperature.

### Example 10

Stability of Estradiol in Compositions

Compositions 1-29 were stored at 25° C. and Relative humidity (RH) 60% for 180 days, and visually observed for any precipitation of estradiol from compositions. No precipitation was observed in Compositions 1-29. In addition to determine physical stability of compositions 1-29 over time, it is necessary to determine if the fill material would be stable during the encapsulation process. One way to test these compositions is with the addition of water to the fill mass. Solubilized estradiol in compositions 1-29 at a concentration of 3.4 mg/g are able to absorb a minimum of 7% water without recrystallization or precipitation. Compositions 1-29 turned hazy on the addition of water.

### Example 11

Analysis of Solubilized Amount of Estradiol and Progesterone

Solubilized amount of estradiol and progesterone in suspension was determined using HPLC assay method. The solubilized amount of estradiol and progesterone in suspension is given in the following table.

TABLE 11
Amounts of Estradiol and Progesterone Solubilized

| S. | Composition | Solubilized amount | Solubilized amount of |
|---|---|---|---|
| No. | No. | of Estradiol in mg | Progesterone in mg |
| 1 | 6 | 0.87 | 13.1 |
| 2 | 7 | 0.97 | 16.0 |
| 3 | 5 | 0.89 | 11.6 |
| 4 | 12 | 0.90 | 9.5 |
| 5 | 13 | 0.89 | 9.4 |
| 6 | 14 | 0.89 | 9.9 |
| 7 | 17 | 0.90 | 9.9 |
| 8 | 18 | 0.89 | 9.9 |
| 9 | 19 | 0.92 | 10.5 |
| 10 | 15 | 0.89 | 9.4 |
| 11 | 24 | 0.94 | 8.4 |
| 12 | 25 | 0.88 | 7.8 |
| 13 | 26 | 0.85 | 7.3 |
| 14 | 19 | 0.89 | 9.2 |

### Example 12

Estradiol and Progesterone combination formulations were prepared, having the compositions were set forth in Table 12.

TABLE 12
Formulations with estradiol and progesterone in combination

| | mg/capsule | | | | |
|---|---|---|---|---|---|
| | Composi- | Composi- | Composi- | Composi- | Composi- |
| Ingredients | tion 30 | tion 31 | tion 32 | tion 33 | tion 34 |
| Estradiol | 1.00 | 1.00 | 1.00 | — | 1.00 |
| anhydrous | | | | | |
| Progesterone | 100.0 | 100.0 | 75 | 75.0 | 100 |
| micronized | | | | | |
| Maisine ® | 141.44 | 188.65 | 115.4 | 115.4 | 115.4 |
| CC | | | | | |
| Kolliphor ® | 45.77 | — | 79.7 | 79.7 | 79.7 |
| RH40 | | | | | |
| Labrafil ® | — | 10.0 | — | — | — |
| M2125 CS | | | | | |
| Anhydrous | 11.44 | — | 4 | 4 | 4 |
| Ethanol | | | | | |
| DL-alpha | 0.35 | 0.35 | 0.35 | 0.35 | 0.35 |
| tocopherol | | | | | |
| Total fill | 300 | 300 | 275.45 | 274.45 | 275.45 |
| composition | | | | | |

Capsule shell composition

| | | | | | |
|---|---|---|---|---|---|
| Gelatin | 1260 | 1260 | 1260 | 1260 | 1260 |
| Hydrolyzed | 150 | 150 | 150 | 150 | 150 |
| Collagen | | | | | |
| Glycerin | 510 | 510 | 510 | 510 | 510 |
| Purified | 1080 | 1080 | 1080 | 1080 | 1080 |
| Water, USP | | | | | |
| FD & C Red | 0.6 | 0.06 | 0.6 | 0.6 | 0.6 |
| No. 40 | | | | | |
| Titanium | 7.5 | 12 | 7.5 | 7.5 | 7.5 |
| dioxide | | | | | |

Batch size equivalent to 20,000 capsules for each composition

Manufacturing Procedure of Composition 30, 32 and 34:

915.4 g and 1594 g and 1594 g of KOLLIPHOR® RH-40 was heated to 60° C. in three containers i.e., Container-1, -2 and -3 respectively and let Kolliphor® RH 40 to reach temperature 30-33° C. 228.8 g, 80 g and 80 g of Ethanol and 7 g of DL-alpha tocopherol were added to Container-1, -2 and -3 respectively at 30-33° C. and stirred until clear solution was obtained. of MAISINE® CC was heated to 40° C. for 5 to 10 minutes and 2828.8 g, 2308 g and 2308 g of MAISINE® CC was added to the above three containers (Container-1; Container-2 and Container-3 respectively) and stirred until clear solutions were obtained. 20 g Estradiol was added to each container and mixed for 30 minutes. 2000 g, 1500 g and 2000 g of micronized progesterone was added to Container-1, -2 and -3 respectively and mixed until uniform dispersion to obtain Composition 30, 32 and 34. These compositions were stored at room temperature until encapsulation.

Manufacturing Procedure of Composition 31:

3773 g MAISINE® CC was taken into a container and was heated to 40° C. 200 g of LABRAFIL® M 2125 CS was added to container and mixed until dissolved completely. To the above solution, 7 g α-tocopherol was added to the container and mixed until clear solution was obtained. estradiol was added to uniform dispersion and mixed for 30 minutes and then 2000 g micronized progesterone was added to the container and mixed to get a uniform dispersion. This final composition was stored at room temperature until encapsulation.

Manufacturing Procedure of Composition 33:

1594 g of KOLLIPHOR® RH-40 was heated to 60° C. in a container and let it to reach temperature 30-33° C. 80 g of Ethanol and 7 g of DL-α-tocopherol were added to the container and stirred until clear solution was obtained. 2308 g of MAISINE® CC was taken in a container and was heated to 40° C. and the added to above clear solution and stirred to get a solution. 1500 g of micronized progesterone was added to the container and mixed to get a uniform dispersion. This final composition was stored at room temperature until encapsulation.

Dissolution Profiles of Composition 30 and 31 in Dissolution Media (3% SDS in 0.1N HCl) at 15 Dips Per Minute (Dpm)

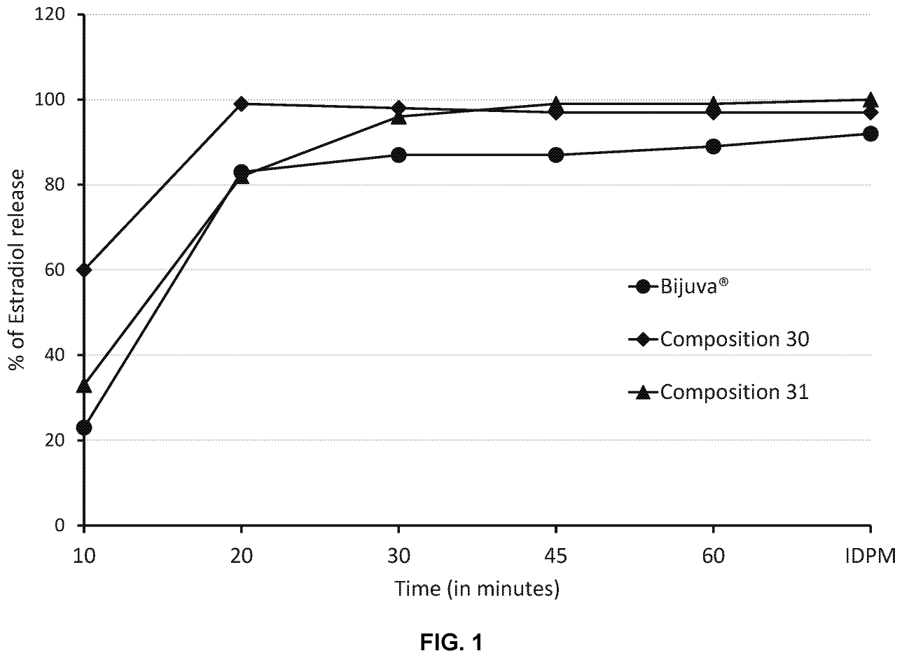

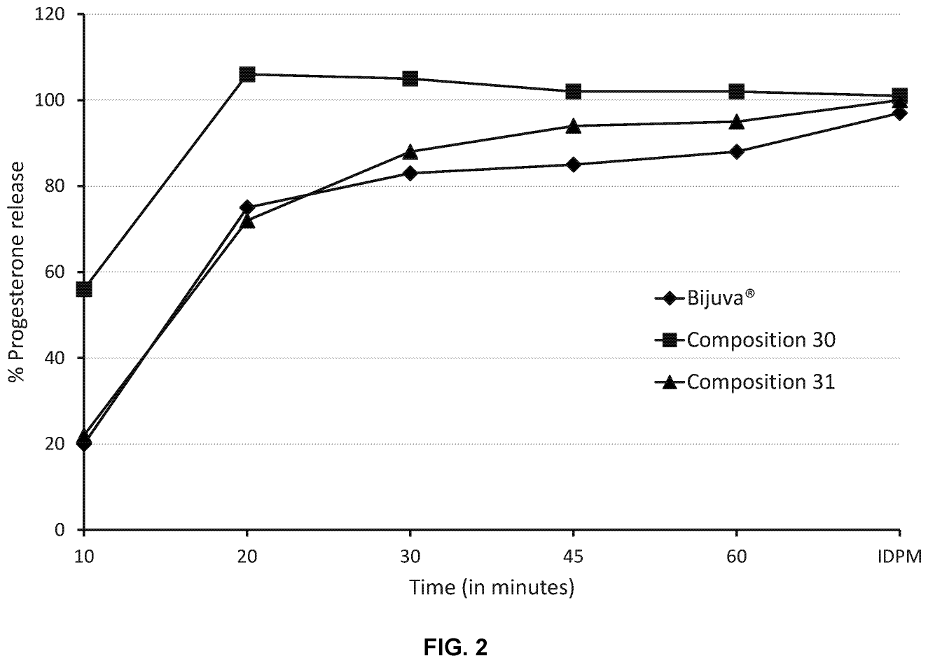

When tested by using USP apparatus III (Reciprocating cylinder; 40 mesh from bottom and top of the inner tube); 250 mL of dissolution media (3% sodium lauryl sulfate in 0.1 N HCl) for 60 minutes at 37±0.5° C. and at 15 dpm (dips per minute), the dissolution profile of Composition 30, 31 was compared with reference composition-1 in following Table 13. Samples of 5 mL were withdrawn at 10, 20, 30, 45 and 60 minutes from dissolution media. Withdrawn samples were filtered with 0.45 μm PTFE filter and then diluted with acetonitrile: water (50:50 v/v) diluent in 2:3 ratio and analyzed using HPLC system with UV spectrophotometer at a wavelength 220 nm. The results of the measurements are given in Table 13 and shown graphically in FIGS. 1 and 2.

TABLE 13
In vitro dissolution release

| | Bijuva ® | | | | | |
|---|---|---|---|---|---|---|
| | (1 mg estradiol & 100 | | | | | |
| | mg progesterone) | | | | | |
| | capsules | | Composition 30 | | Composition 31 | |
| | % | % | % | % | % | % |
| Time | Estradiol | Progesterone | Estradiol | Progesterone | Estradiol | Progesterone |
| (minutes) | release | release | release | release | release | release |
| 10 | 23 | 20 | 60 | 56 | 33 | 22 |
| 20 | 83 | 75 | 99 | 106 | 82 | 72 |
| 30 | 87 | 83 | 98 | 105 | 96 | 88 |
| 45 | 87 | 85 | 97 | 102 | 99 | 94 |
| 60 | 89 | 88 | 97 | 102 | 99 | 95 |
| IDPM* | 92 | 97 | 97 | 101 | 100 | 100 |

IDPM — infinite point (50 dpm for 30 minutes)

### Example 13

The evaluation of phase separation for binary mixtures at different ratios was conducted, and is summarized in Table 14.

TABLE 14
Evaluation of phase separation

| | | Day- | Day- | Day- | Day- |
|---|---|---|---|---|---|
| Sample | Binary mixture | 1 | 2 | 3 | 4 |
| 1 | Capmul MCM:Gelucire ® 44/4 | NPS | NPS | NPS | NPS |
| | (98:2) | | | | |
| 2 | Maisine ® CC:Kolliphor ® RH40 | PS | PS | PS | PS |
| | (98:2) | | | | |
| 3 | Maisine ® CC:Kolliphor ® RH40 | PS | PS | PS | PS |
| | (95:5) | | | | |
| 4 | Maisine ® CC:Kolliphor ® RH40 | PS | PS | PS | PS |
| | (93:7) | | | | |
| 5 | Maisine ® CC:Kolliphor ® RH40 | NPS | PS | PS | PS |
| | (90:10) | | | | |
| 6 | Maisine ® CC:Kolliphor ® RH40 | NPS | NPS | NPS | NPS |
| | (88:12) | | | | |
| 7 | Maisine ® CC:Kolliphor ® RH40 | PS | PS | PS | PS |
| | (85:15) | | | | |

Note:
NPS is No Phase Separation;
PS is Phase Separation

### Example 14

Compositions which were stable for 8 days after addition of excess water were set forth in Table 15.

TABLE 15
Stability testing

| | Ingredients | | | | | |
|---|---|---|---|---|---|---|
| | | | | Kolliphor | | Physically |
| Composition No. | Estradiol | Progesterone | Maisine | RH40 | Ethanol | stable |
| 30 | 1 mg | 100 mg | 70 | 24 | 6 | 8 days |
| 31 | 1 mg | 100 mg | 68.75 | 25 | 6.25 | 8 days |
| 32 | 1 mg | 100 mg | 67.50 | 26 | 6.5 | 8 days |
| 33 | 1 mg | 100 mg | 66.25 | 27 | 6.75 | 8 days |
| 34 | 1 mg | 100 mg | 65 | 28 | 7 | 8 days |
| 35 | 1 mg | 100 mg | 60 | 32 | 8 | 8 days |
| 36 | 1 mg | 100 mg | 50 | 40 | 10 | 8 days |
| 37 | 1 mg | 100 mg | 50 | 35 | 15 | 8 days |
| 38 | 1 mg | 100 mg | 50 | 30 | 20 | 8 days |
| 39 | 1 mg | 100 mg | 50 | 25 | 25 | 8 days |

### Example 15

In-vitro lipid digestion studies were carried out as described below.

TABLE 16
Composition of simulated intestinal medium

| Ingredients | Quantity | |
|---|---|---|
| Bile salts (mM) | 5.0 | mM |
| Lecithin (mM) | 1.25 | mM |
| Trizma-maleate (mM) | 2 | mM |
| Na⁺ (mM) | 150 | mM |
| Pancreatic lipase (USP units/mL) | 800 | USP units/mL |

| | |
|---|---|
| Ca⁺² (mmol/minute) | 0.045 (0.09 mL/min of |
| | 0.5M Ca+2 solution |

Preparation of pancreatin solution: 17.50 g of pancreatin was added to 110 mL of milliQ water at 37° C., stirred well and centrifuged for 7 min at 4000 rpm before the pH was adjusted to 6.5.

The simulated intestinal medium (200 mL) was added to each thermostated jacketed glass vessel-1, vessel-2 and vessel-3 with paddles stirring and equilibrated to 37° C. and pH 6.5 for approximately 5 minutes. Then 300 mg of the Composition 9, Composition 26 and Reference composition-1 were added to vessel-1 and vessel-2 respectively and left to equilibrate for additional 10 minutes to ensure complete dispersion of the compositions. The lipolysis was started and after 2 minutes, 100 mL of the pancreatic solution was added to the vessel-1 and vessel-2.

Throughout the experiment (120 minutes), the temperature was kept at 37° C. and the pH was continuously adjusted to 6.5 through addition of 1.0 M NaOH using a pH-Stat 902 Titrando from Metrohm (Herisau, Switzerland). In addition, 0.045 mmol/min of the Ca⁺² solution was continuously added during the experiment.

TABLE 17
Parameter settings

| Pre. pH calibration | 120 | sec |
|---|---|---|
| Lipolysis stop time | 7200 | sec |
| Stirring rate | 10 | rpm |
| End point | 6.5 | pH |
| Dosing rate (1.0M NaOH) | Maximum mL/minute | |
| Dosing rate (0.5M CaCl₂) | 0.09 | mL/minute |
| Temperature | 37° | C. |

Six homogenous samples were taken during the experiment: 1 minute (before initiation of lipolysis), 7 minutes (after 5 minutes of lipolysis), 17 minutes (after 15 minutes of lipolysis), 32 minutes (after 30 minutes of lipolysis), 62 minutes (after 60 minutes of lipolysis), 120 minutes (after 118 minutes of lipolysis).

Sample preparation: A homogenous sample of 250 μL was pipetted directly into a 1.5 mL Eppendorf tube containing 1000 μL acetonitrile and 225 μL 0.5 M HCl. This was subsequently centrifuged at 10,000 rpm for 5 min and the supernatant was analyzed for progesterone content using HPLC-UV.

Solubilized drug sample: A homogenous sample of 1000 μL was added to 25 μL 4-bromobenzene boronic acid solution (1 M in methanol; enzyme inhibitor), and subjected to ultracentrifugation (30 min at 100,000 rpm, 37° C.) in an Optima MAXXP ultracentrifuge (Beckman Coulter, Brea, Calif., USA).

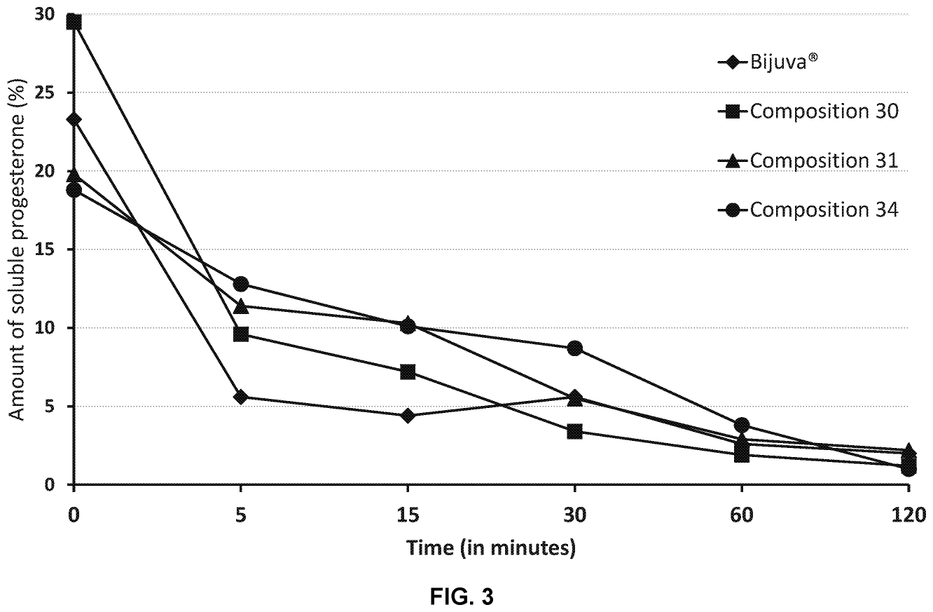

Subsequently, 250 μL of the supernatant was pipetted into a 1.5 mL Eppendorf tube containing 1000 μL acetonitrile and 225 μL 0.5 M HCl. The Eppendorf tube was centrifuged at 10,000 rpm for 5 minutes and the supernatant analyzed for progesterone content using HPLC-UV. All formulations were tested in triplicate (n=3). The results of the measurements are given in Table 18 and shown graphically in FIG. 3.

TABLE 18
in-vitro lipid digestion studies data

| | Amount of soluble progesterone | | | |
|---|---|---|---|---|
| | (%, relative to total sample) | | | |
| Time | | Composition | Composition | Composition |
| (in minutes) | Bijuva ® | 30 | 31 | 34 |
| 0 | 23.3 | 29.5 | 19.8 | 18.8 |
| 5 | 5.6 | 9.6 | 11.4 | 12.8 |
| 15 | 4.4 | 7.2 | 10.3 | 10.1 |
| 30 | 5.6 | 3.4 | 5.5 | 8.7 |
| 60 | 2.6 | 1.9 | 2.9 | 3.8 |
| 120 | 2 | 1.2 | 2.2 | 1 |

### Example 16

Evaluation of impurities formed in Composition 30 and 31, when stored at 40° C. and 75% RH for three months, was conducted and summarized in Tables 19 and 20.

TABLE 19
Related substance data of progesterone

| | | | | Impurities (%) | | | |
|---|---|---|---|---|---|---|---|
| | | | | Composition 30 | | Composition 31 | |
| | | | Specification | | 40° C. & | | 40° C./ |
| | Name of the | | (as per ICH) | | 75% RH | | 75% RH |
| S. No. | Impurity | RRT | % Impurity | Initial | 3 months | Initial | 3 months |
| 1 | 6-Beta Impurity | 0.35 | 0.20 | ND | ND | ND | ND |
| 2 | 6-Oxo Impurity | 0.55 | 0.20 | 0.008 | 0.009 | 0.009 | 0.008 |
| 3 | Impurity-6 | 0.65 | 0.20 | ND | ND | ND | ND |
| 4 | Impurity-C | 0.95 | 0.20 | ND | ND | ND | ND |
| 5 | Impurity-K | 0.88 | 0.20 | 0.022 | 0.027 | 0.028 | 0.028 |
| 6 | Impurity-M | 1.07 | 0.20 | ND | 0.016 | ND | 0.020 |
| 7 | Impurity-I | 1.99 | 0.60 | 0.333 | 0.348 | 0.349 | 0.361 |
| 8 | Single Max | 2.10 | 0.20 | 0.052 | 0.059 | 0.057 | 0.063 |
| | Unknown | | | | | | |
| 9 | Total Impurities | NA | 2.00 | 0.449 | 0.490 | 0.475 | 0.512 |

TABLE 20
Related substance data of estradiol

| | | | | Impurities (%) | | | |
|---|---|---|---|---|---|---|---|
| | | | | Composition 30 | | Composition 31 | |
| | | | Specification | | 40° C. & | | 40° C./ |
| | | | (as per ICH) | | 75% RH | | 75% RH |
| S. No. | Name of the Impurity | RRT | % Impurity | Initial | 3 months | Initial | 3 months |
| 1 | 6-Alpha | 0.35 | 0.20 | ND | ND | ND | ND |
| | Estradiol | | | | | | |
| 2 | 6-Keto Estradiol | 0.55 | 0.20 | 0.008 | 0.009 | 0.009 | 0.008 |
| 3 | Delta-6-Estradiol | 0.65 | 0.20 | ND | ND | ND | ND |
| | (RC-B) | | | | | | |
| 4 | Delta-9,11- | 0.95 | 0.20 | ND | ND | ND | ND |
| | Estradiol | | | | | | |
| | (EP Imp-D) | | | | | | |
| 5 | Estrone | 0.88 | 0.20 | 0.022 | 0.027 | 0.028 | 0.028 |
| | (EP-RC-A) | | | | | | |
| 6 | Single Max | 1.07 | 0.20 | ND | 0.016 | ND | 0.020 |
| | Unknown | | | | | | |
| 7 | Total Impurities | 1.99 | 0.60 | 0.333 | 0.348 | 0.349 | 0.361 |
| 8 | 6-Alpha | 2.10 | 0.20 | 0.052 | 0.059 | 0.057 | 0.063 |
| | Estradiol | | | | | | |
| 9 | Total Impurities | NA | 2.00 | 0.449 | 0.490 | 0.475 | 0.512 |

Having now fully described this invention, it will be understood by those of ordinary skill in the art that it can be performed within a wide equivalent range of parameters without affecting the scope of the invention or any embodiment thereof. All publications, patent applications and patents disclosed herein are incorporated by reference in their entirety.

1. A stable pharmaceutical composition suitable for oral administration to a subject in need thereof, comprising:
progesterone or a pharmaceutically acceptable salt thereof;
optionally, estradiol or a pharmaceutically acceptable salt thereof;
a solubilizing agent comprising a long-chain oil;
a surfactant;
optionally, an antioxidant; and
optionally a co-solvent.

2. A stable pharmaceutical composition suitable for oral administration to a subject in need thereof, comprising:
progesterone or a pharmaceutically acceptable salt thereof;
estradiol or a pharmaceutically acceptable salt thereof;
a solubilizing agent comprising a long-chain oil;
a surfactant;
optionally, an antioxidant; and
optionally a co-solvent.
wherein the pharmaceutical composition upon oral administration exhibits bioequivalence to reference composition-1.

3. A stable pharmaceutical composition suitable for oral administration to a subject in need thereof, comprising:
progesterone or a pharmaceutically acceptable salt thereof;
estradiol or a pharmaceutically acceptable salt thereof;
a solubilizing agent comprising a long-chain oil;
a surfactant;
optionally, an antioxidant; and
optionally a co-solvent.
wherein the pharmaceutical composition upon oral administration exhibits increased bioavailability of progesterone when compared with reference composition-1.

4. A stable pharmaceutical composition suitable for oral administration to a subject in need thereof, comprising:
progesterone or a pharmaceutically acceptable salt thereof;
a solubilizing agent comprising a long-chain oil;
a surfactant;
optionally, an antioxidant; and
optionally a co-solvent.
wherein the pharmaceutical composition upon oral administration exhibits increased bioavailability when compared with reference composition-2.

5. The stable pharmaceutical composition according to claim 1, wherein the long-chain oil comprises at least one of C₁₄-C₂₄ fatty acid mono, di or tri-esters of glycerol or mixtures thereof.

6. The stable pharmaceutical composition according to claim 1, wherein the long-chain oil comprises at least one of C₁₆-C₁₈ fatty acid mono, di or tri-esters of glycerol or mixtures thereof.

7. The stable pharmaceutical composition according to claim 1, wherein an average HLB value of the surfactant(s) is from about 9 to about 20.

8. The stable pharmaceutical composition according to claim 1, wherein the solubilizing agent is selected from the group consisting of a glyceryl mono-myristate, a glyceryl mono-palmitate, a glyceryl mono-oleate, a glyceryl dioleate, a glyceryl dioleate, a glyceryl mono-linoleate, a glycerol mono-stearate, a glyceryl palmitic/stearic, a glyceryl mono-α-linolenic acid, a glyceryl mono-elaidate, a glyceryl mono-vaccenate, a glyceryl mono-linoelaidate, a glyceryl mono-arachidonate, a glyceryl mono-eicosapentaenoate, a glyceryl mono-erucic acid, a glyceryl mono-docosahexaenoic acid, and mixtures thereof.

9. The stable pharmaceutical composition according to claim 1, wherein the solubilizing agent and surfactant are present in the composition in a weight ratio of about 50:50 to about 99:1, preferably about 60:40 to about 99:1.

10. The stable pharmaceutical composition according to claim 1, wherein the co-solvent is selected from the group consisting of ethanol, polyethylene glycol, propylene glycol, and mixtures thereof.

11. The stable pharmaceutical composition according to claim 1, wherein the co-solvent is ethanol.

12. The stable pharmaceutical composition according to claim 1, wherein the progesterone or a pharmaceutically acceptable salt thereof is a sole active ingredient.

13. The stable pharmaceutical composition according to claim 1, wherein the progesterone or a pharmaceutically acceptable salt thereof, and the estradiol or a pharmaceutically acceptable salt thereof, are both present as active ingredients.

14. The stable pharmaceutical composition according to claim 1, wherein the progesterone or a pharmaceutically acceptable salt thereof is present in an amount from about 30 mg to about 150 mg.

15. The stable pharmaceutical composition according to claim 12, wherein the composition provides increased progesterone bioavailability compared with reference composition-2 in a fed state or a fasted state.

16. The stable pharmaceutical composition according to claim 13, wherein the composition provides increased progesterone bioavailability compared to reference composition-1 in a fed state or a fasted state.

17. The stable pharmaceutical composition according to claim 1, wherein when the composition is stored for 6 months at 40° C./75% relative humidity (RH), the level of Impurity-M in the composition is less than about 0.2% (w/w) as measured by HPLC.

18. The stable pharmaceutical composition according to claim 1, wherein the co-solvent is present in an amount sufficient to inhibit phase separation for at least 24 hours when stored at 25±2° C. and 60±5% relative humidity (RH).

19. The stable pharmaceutical composition according to claim 1, wherein not less than 80% of the progesterone is released after about 45 minutes as determined using USP Apparatus III at 15 dpm in 3% SLS in 0.1 N HCl dissolution media at 37° C.

20. A method for treating vasomotor symptoms (VMS) related to menopause in a human patient, which method comprises administering a pharmaceutical composition according to claim 1.

21. A method for treating secondary amenorrhea in a human patient, which method comprises administering a pharmaceutical composition according to claim 4.
